SEQUENCE LISTING (8 sequences)

SEQ ID NO: 1 - DNA, 2250 bp, Homo sapiens
atggatataactgatatcaacactccaaagccaaagaagaaacagcgatggactccactggagatcagcctctcggtccttgtcctgctcctcaccatcatagctgtgacaatgatcgcactctatgcaacctacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtgatcttcaaaagaagcattg

SEQ ID NO: 2 - protein, 743 aa, Homo sapiens
MDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 3 - DNA, 5634 bp, Homo sapiens
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtaataaccgcggcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 4 - protein, 782 aa, Homo sapiens
MRFPSIFTAVLFAASSALAAPVNTTTEDETAQIPAEAVIGYSDLEGDFDVAVLPFSNSTNNGLLFINTTIASIAAKEEGVSLEKRDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 5 - DNA, 5633 bp, Homo sapiens
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtatacagaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgcccaggctacggctaaacctgaagatcgaaatgatccaatgcttctgtataacaagatgacattggcccagatccaaaatcagttttcactagagatcaatgggaagccattcagctggttgcagttcacaaatgaaatcatgtcaactgtgcagattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggacagttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtaataaccgcggcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 6 - protein, 21 aa, Homo sapiens
DRLIEDICLPRWGCLWEDDGS

SEQ ID NO: 7 - DNA, 5697 bp, Homo sapiens
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaccgcctgatcgaggatatctgcctgccccggtggggctgcctgtgggaggatgatggtagtgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtaataaccgcggcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 8 - DNA, 5697 bp, Homo sapiens
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaccgcctgatcgaggatatctgcctgccccggtggggctgcctgtgggaggatgatggtagtgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtatacaggaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgcccaggctacggctaaacctgaagatcgaaatgatccaatgcttctgtataacaagatgacattggcccagatccaaaatcagttttcactagagatcaatgggaagccattcagctggttgcagttcacaaatgaaatcatgtcaactgtgcagattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggacagttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtaataaccgcggcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

(19) United States
(12) Patent Application Publication
(10) Pub. No.: US 2006/0029590 A1
(43) Pub. Date: Feb. 9, 2006

(54) Administration of neutral endopeptidase to treat inflammatory bowel disease

(76) Inventors: Christopher Thanos, San Francisco, CA (US); Edwin Madison, South San Francisco, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/149,513

(22) Filed: Jun. 10, 2005

Related U.S. Application Data
(60) Provisional application No. 60/578,911, filed on Jun. 10, 2004.

(51) Int. Cl. A61K 38/48; C12P 21/06; C12N 9/64

(52) U.S. Cl. 424/94.63; 435/69.1; 435/226; 435/320.1; 435/325

(57) ABSTRACT

Administration of recombinant, truncated mammalian NEP or certain bacterial homologues of this protein is therapeutically effective in the treatment of inflammatory bowel disease.

### RELATED APPLICATION

This application claims priority to provisional patent application U.S. Ser. No. 60/578,911, filed Jun. 10, 2004, which is incorporated herein by reference in its entirety.

### BACKGROUND OF THE INVENTION

1. Field of the Invention

The present invention generally relates to methods, preparations and pharmaceutical compositions for treating or preventing inflammatory diseases in mammalian subjects.

2. Description of Related Disclosures

Tachykinins are a family of neuropeptides that are widely expressed in the nervous system (Otsuka et al., 1993, and McDonald et al., 1996. A list of references cited is located at the end of this specification; all references cited herein are incorporated herein by reference). Notably, tachykinins are expressed by primary spinal afferent neurons and the enteric nervous system. Inflammatory stimuli trigger the release of substance P (SP) from the peripheral projections of primary spinal afferent neurons. SP activates neurokinin receptors (notably NKIR) on endothelial cells and immune cells to induce inflammation of peripheral tissues (neurogenic inflammation) (McDonald et al., 1996). Bradykinin is generated locally at sites of inflammation. Bradykinin stimulates SP release and also exerts direct inflammatory effects by activating the bradykinin type 2 receptors.

There are multiple receptors for the tachykinins, including the NK1, NK2, and NK3 receptors, as well as others. Involvement of tachykinins in inflammatory bowel disease (IBD) in humans is illustrated by observations that the NK1R is markedly up-regulated on arterioles, venules, lymph nodes and muscle cells in patients with IBD (Manyth et al., 1995 and 1998) and that SP levels are elevated in patients with ulcerative colitis. There are similar alterations in NK1R expression and SP levels in animal models of IBD (Mantyh et al., 1996), and in some models, antagonism of the NK1R prevents inflammation (Pothoulakis C, 1994, and Sturiale, 1999). Upon trauma and inflammation, a cascade of these pro-inflammatory peptides is released and bound by these receptors. To down-regulate this response, an antagonist has to overcome this cascade, which possesses built-in redundancies. Conventional small molecule or mAb (monoclonal antibody) therapeutic modalities, which typically have a 1:1 stoichiometry, are therefore not well suited for this type of therapeutic target. There remains a need for a therapeutic agent with broad specificity for hydrolysis of tachykinins to overcome the redundancy of the cascade and down-regulate the response so as to achieve a therapeutic effect.

In animals, NEP (neutral endopeptidase) is a cell-surface enzyme that degrades several biologically active peptides that mediate inflammation, notably tachykinins and bradykinin. From a kinetic standpoint, Substance P is the most favorable substrate. Deletion of NEP or administration of NEP inhibitors results in diminished degradation of SP and bradykinin and elevated tissue levels of these peptides. Animals lacking NEP exhibit exacerbated inflammation of the small intestine (Kirkwood et al., 2001), colon (Sturiale et al., 1999), pancreas, and skin (Scholzen et al., 2001), which can be attenuated by administration of recombinant human NEP or of antagonists of the NK1R. Moreover, NEP levels are markedly diminished in the inflamed intestine of rats (Scholzen et al., 2001) and humans, which may exacerbate inflammation. While there has been speculation that replenishment of NEP levels might be a viable mode of therapeutic intervention (see U.S. Pat. Nos. 5,262,178; 5,403,585; and 5,780,025, each of which is incorporated herein by reference), a number of problems have prevented any realization of this potential therapeutic modality.

First, NEP is a large, transmembrane domain containing, multidomain, heavily glycosylated protease expressed in multiple tissues in the body (Roques et al., 1993). The DNA sequence encoding rat and human NEP is disclosed in U.S. Pat. No. 4,960,700, incorporated herein by reference. In its natural form, NEP is not suitable as a therapeutic, because it is not only difficult to express but also possesses a hydrophobic transmembrane domain that makes it highly unlikely that the intact protein would be a useful therapeutic. While there has been speculation that truncated forms of the protein might be useful, such truncated forms have likewise proven to be difficult to express. Thus, any promise there might have been that NEP proteins could be used to treat disease has not been realized, and there remains a need for new therapeutically effective agents to treat inflammation, particularly inflammatory bowel disease. The present invention helps meet these and other needs by providing methods for making and purifying truncated forms of NEP and for treating diseases with pharmaceutical compositions of the invention that comprise them.

### SUMMARY OF THE INVENTION

In a first aspect, the present invention provides materials and methods for expressing recombinant, truncated mammalian NEPs and NEP homologs from bacteria and for purifying them to homogeneity such that pharmaceutical compositions comprising them can be prepared. As used herein, “truncated” means that a portion, and in preferred embodiments all, of the transmembrane domain has been deleted from the NEP, relative to a wild-type NEP. In one embodiment, the invention provides recombinant DNA expression vectors suitable for producing a truncated NEP in a yeast cell, and methods for producing the NEP in large amounts in yeast. In one embodiment the yeast is Pichia pastoris.

The invention provides a method for making a recombinant, truncated mammalian neutral endopeptidase (NEP) by culturing a host cell that includes a nucleic acid vector encoding a truncated mammalian NEP, such as amino acids 47-749 of SEQ ID NO: 2. For example, the vector is pPicZα-A-NEP. The vector is integrated into the host cell genome. Alternatively, the vector is not integrated into the host cell genome, but remains episomal. The host cell includes mammalian cells (e.g., human cells), non-mammalian eukaryotic cells, and prokaryotic cells such as bacteria.

The invention also provides a method for purifying a recombinant, truncated mammalian neutral endopeptidase (NEP) by adding ammonium sulfate (e.g., to generate a 60% solution) to a solution comprising said NEP, and subjecting the solution to chromatography comprising hydrophobic interaction chromatography and anion exchange chromatography. In embodiments, the method also includes the step of removing any precipitate, if present, from the solution following addition of the ammonium sulfate. The invention also provides the purified recombinant, truncated mammalian NEP obtained by this method. In certain embodiments, the purified NEP is more than 80%, 85%, 90%, 92%, 95%, 96%, 97%, 98%, 99%, 99.9%, or 99.99% pure. In embodiments, the truncated mammalian NEP is essentially free from lypopolysaccharides. “Essentially free from” or “essentially pure” when used to describe the state of NEPs produced by the invention means free of protein or other materials normally associated with NEP in its in vivo physiological milieu, as for example when NEP is obtained from blood and/or tissues by extraction and purification.

In a second aspect, the present invention provides pharmaceutical compositions comprising a truncated NEP useful in the treatment of a disease or disease condition. In one embodiment, the NEP is a human NEP. In another embodiment, the NEP is enzymatically deglycosylated in order to increase the in-vivo half life of the protein. In another, the NEP has each of six asapargines that correspond to mapped N-linked glycosylation sites (with the N-X-S/T glycosylation signature) mutated to one of the 19 other amino acids in order to prevent the protein from being naturally glycosylated during expression. In another, the NEP is a rodent NEP, including but not limited to rat, hamster, and mouse NEP. In another, the NEP is an NEP homolog from a bacterium. In one embodiment, the bacterial NEP homolog is identical or homologous to the NEP homolog from the benign intestinal bacteria Lactococcus lactis. By way of non-limiting example, mammalian NEP homologs include Accession numbers: P08473, P07861, Q61391, P08049, P42891, P42893, P42892, P97739, O60344, P78562, P70669, Q10711, O95672, Q9JMI0, Q9JHL3, Q22523, O52071, P23276, P42359, Q07744, Q09145, Q09319, Q9X5U8, and P89876; bacterial and nematode NEP homologs include Q07744, Q09145, O52071, P42359, P97739, and Q22523.

The invention also provides pharmaceutical composition in a unit dose. For example, one unit dose contains from about 1 to about 200 mg of a truncated NEP or an NEP homolog, and a pharmaceutically acceptable excipient or carrier, or other amounts of truncated NEP, such as between 20 and 100 mg of said truncated NEP or NEP homolog.

The invention also provides compositions containing NEP muteins. By way of non-limiting example, an NEP mutein has reduced N-linked glycosylation, and contains amino acids 47-749 of SEQ ID NO: 2, wherein one or more asparagine residues in SEQ ID NO: 2 are replaced by one or more amino acids other than asparagine. The invention also provides a pharmaceutical formulation comprising recombinant, truncated mammalian neutral endopeptidase (NEP) comprising amino acids 47-749 of SEQ ID NO: 2. In embodiments, the pharmaceutical formulation is encapsulated in an enteric coating.

In a third aspect, the present invention provides methods for treating an inflammatory disease, such as Inflammatory Bowel Disease (IBD) or a symptom thereof by administering a therapeutically effective dose of a pharmaceutical composition comprising a truncated NEP.

The invention also provides methods of treatment of inflammatory bowel disease in a human patient suffering therefrom by administering to the human a unit dose of truncated NEP, wherein the unit dose consists of between 20 and 100 mg of said truncated NEP or NEP homolog. The administration can by by infusion (e.g., intraveneous infusion) or by other means such that the NEP is delivered to the target cell, tissue or organ. In certain embodiments, the NEP contains amino acids 47-749 of SEQ ID NO: 2. Alternatively, the NEP contains amino acids 47-749 of SEQ ID NO: 2, wherein one or more asparagine residues in SEQ ID NO: 2 are replaced by one or more amino acids other than asparagine. In an embodiment, the NEP contains amino acids 47-749 of SEQ ID NO: 2, wherein N144, N284, N310, N324, N334, and N627 are replaced by one or more amino acids other than asparagine.

The invention further provides a method for treating inflammatory bowel disease in a mammalian subject in need thereof by administering to the subject a therapeutically effective dose of a recombinant, truncated mammalian neutral endopeptidase (NEP) or a bacterial homolog of said NEP. For example, the NEP contains amino acids 47-749 of SEQ ID NO: 2. The NEP is administered at a dose ranging from 0.01 mg of NEP per kg of said subject's body weight to 100 mg/kg of NEP per kg of said subject's body weight, such as between 0.1 mg/kg and 10 mg/kg, between 0.5 mg/kg and 5 mg/kg, and between 1 mg/kg and 2 mg/kg.

The invention also provides a method for preventing or reducing a symptom of inflammatory bowel disease in a mammalian subject by identifying a mammalian subject at risk of inflammatory bowel disease, and administering to the subject a therapeutically effective dose of a recombinant, truncated mammalian neutral endopeptidase (NEP) or a bacterial homolog thereof. The subject may be identified based upon the subject's prior history of IBD, the subject's genetic profile, or the subject's family history of IBD. In some embodiments, the NEP is administered to the subject prior to the onset of one or more symptoms of inflammatory bowel disease. Alternatively, the NEP is administered to the subject at the onset of one or more symptoms of inflammatory bowel disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE FIGURES

FIG. 1 is a photograph of Pichia pastoris X33 cells growing on YPDS plates containing 500 ug/mL of zeocin in the growth medium.

FIG. 2 is a photograph of a protein gel containing samples of recombinant, truncated human NEP produced by the P. pastoris expression system of the invention, after elution from a hydrophobic interaction column in accordance with the purification method of the invention.

FIG. 3 shows the results of this assay from a typical test. In the figure, “CAT-NEP” is a recombinant, truncated NEP prepared in accordance with the methods of the invention; “LAC-NEP” is a homolog of NEP cloned from the benign intestinal bacterium Lactococcus lactis; “Buffer” is 50 mM MES, pH 6.4, and serves as a non-NEP containing negative control for the assay.

FIG. 4 is a gel showing deglycosylated NEP vs. fully glycosylated NEP. Enzymatic deglycosylation results in a decrease in MW by approximate 10 KDa.

FIG. 5 is an electrospray mass spec analysis of enzymatically deglycosylated NEP which shows that each of putative N-linked glycosylation sites on NEP is modified by a residual glc-nac hexose, verifying that each site is indeed glycosylated in vivo. The 6 peaks at 80349, 80449, 80553, 80657, 80755, and 80854 correspond to different modification states of NEP by the hexose glc-nac.

FIG. 6 is a figure showing that the in-vivo half life of enzymatically degraded NEP is 10-fold greater than the fully glycosylated form.

FIG. 7 is a figure showing that recombinant deglycosylated NEP can protect against weight loss TNBS induced colitis when administered topically.

FIG. 8 is a figure showing that recombinant deglycosylated NEP can protect mice from TNBS induced colitis as measured by gross score, edema score, and histopathology score.

FIG. 9 is a figure showing that recombinant deglycosylated NEP can protect mice from TNBS induced colits as measured by examination of gross colon morphology.

FIG. 10 is a figure showing that recombinant deglycosylated NEP can treat severe colitis in the IL-10 knockout model at the following doses. “Low Dose” corresponds to 8 mg/kg/day and “High Dose” corresponds to 24 mg/kg/day.

FIG. 11 is a figure showing that recombinant deglycosylated NEP can treat mild colits in the IL-10 knockout at the following doses: “Low Dose” corresponds to 8 mg/kg/day and “High Dose” corresponds to 24 mg/kg/day.

FIG. 12 is a figure showing colon histology in treated vs non-treated severe colitis in IL-10KO model.

### DETAILED DESCRIPTION OF THE INVENTION

The present invention discloses methods and compositions for treating inflammatory disorders in humans or other mammals, including Inflammatory Bowel Disease (IBD). IBD is also termed Crohns' Disease, ileitis or enteritis. Symptoms of IBD include abdominal pain, diarrhea or constipation or alternating diarrhea and constipation, gas, bloating, nausea, weight loss, rectal bleeding, fatigue, and decreased appetite. Children suffering from IBD also experience delayed growth and development. Subjects suffering from IBD have symptoms similar to subjects suffering from Irritable Bowel Disease (also known as Irritable Bowel Syndrome) or ulcerative colitis.

IBD frequently causes inflammation in the small intestine, e.g., the lower part of the small intestine, called the ileum, but it can affect any part of the digestive tract, from the mouth to the anus. The inflammation extends deep into the lining of the affected organ. The inflammation can cause pain and can make the intestines empty frequently, resulting in diarrhea. IBD is generally a chronic disorder. (See, digestive.niddk.nih.gov/ddiseases/pubs/crohns).

In IBD, a severe ulceration of the intestinal lumen is often observed. In one aspect of this invention, direct subluminal administration of an NEP-containing pharmaceutical composition to the site of inflammation is employed to treat IBD. Other methods of administration are also provided by the invention. The invention provides methods for administering the NEP-containing pharmaceutical composition by encapsulated oral delivery, direct injection to the bowels, anal suppository, and enema to treat diseases involving intestinal inflammation.

The present invention also provides a variety of recombinant, mammalian truncated NEPs for use as therapeutically effective agents in the treatment of intestinal inflammation. In one embodiment, the NEP of the invention is the recombinant human NEP described in Example 1 below. The present invention also provides expression vectors and methods for purifying a recombinant mammalian NEP, as described in Examples 1 and 2 below.

In addition, the present invention provides other modified forms of mammalian NEPs useful in the treatment of intestinal inflammation. There are a number of loops on the surface of NEP (Protein Data Bank Accession Code: 1DMT) that are solvent accessible and constrained by alpha helices at both ends. In human NEP, these loops span from residues 71-82, 93-102, 259-265, 333-342, 668-681, and 732-749. Peptides that bind with high affinity to a serum protein or proteins of the vasculature, such as albumin, platelet receptors, cell surface proteins, antibodies, or soluble blood proteins are placed within one or more of these loops to provide novel NEPs of the invention that have, relative to the truncated NEP described in Example 1, increased serum half life and/or are more stable without detrimentally affecting the selectivity or the activity of NEP. Methods for identifying such peptides include but are not limited to phage display, ribosome display, peptides on plasmids, and the like.

The present invention also provides bacterial homologs of truncated, mammalian NEP useful in the methods and compositions of the present invention. In one embodiment, the bacterial homolog is identical or homologous to the NEP homolog from the benign intestinal bacteria Lactococcus lactis. Bacterial NEP homologs of the invention can be prepared using recombinant DNA methodology and by isolation from cultures of a recombinant or naturally occurring strain of a producing bacterium.

A truncated NEP polypeptide of the invention includes for example, a protein containing amino acids 47-749 of SEQ ID NO: 2. Alternatively, a truncated NEP polypeptide contains amino acids 41-749, 42-749, 43-749,44-749, 45-749, or 46-749 of SEQ ID NO: 2. The invention also provides a “mutein,” which is a mutant or variant protein any of whose residues may be changed from the corresponding residue shown in SEQ ID NO:2 while still encoding a protein that maintains its NEP-like activities and physiological functions, or a functional fragment thereof. In some embodiments, up to 20% or more of the residues may be so changed in the mutant or variant protein. Preferably, the NEP mutein is at least about 80% homologous to wild-type NEP, more preferably at least about 85%, 90%, 95%, 98%, and most preferably at least about 99% homologous to wild-type NEP. In general, an NEP variant that preserves NEP function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. Amino acid substitutions are typically of single residues; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined. Obviously, the mutations that will be made in the DNA encoding the NEP mutein should not place the sequence out of reading frame and preferably will not create complementary regions that could produce secondary mRNA structure. In favorable circumstances, the substitution is a conservative substitution. For example, nucleotide substitutions leading to amino acid substitutions at “non-essential” amino acid residues can be made in the sequence of SEQ ID NO: 1. A “non-essential” amino acid residue is a residue that can be altered from the wild-type sequence of NEP without altering the biological activity, whereas an “essential” amino acid residue is required for biological activity. For example, amino acid residues that are conserved among the NEP proteins of the present invention, are predicted to be particularly unamenable to alteration.

NEP muteins also contain one or more insertions, deletions, or substitutions of an amino acid while still having substantially similar activity of a NEP polypeptide. In embomdiments, substitutions are made in accordance with Table S1, below. See also U.S. Pat. No. 5,780,025, which is incorporated by reference herein in its entirety.

| Original residue | Exemplary substitution(s) |
|---|---|
| Ala | Ser |
| Arg | Lys |
| Asn | Gln, His |
| Asp | Glu |
| Cys | Ser |
| Gln | Asn |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those in Table S1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce changes in NEP properties will be those in which (a) a hydrophilic residue, e.g. seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

While the site for introducing an amino acid sequence variation is predetermined, the mutation per se need not be predetermined. For example, in order to optimize the performance of a mutation at a given site, random mutagenesis may be conducted at the target codon or region and the expressed NEP muteins screened for the optimal combination of desired activity. Techniques for making substitution mutations at predetermined sites in DNA having a known sequence are well known, for example M13 primer mutagenesis.

Therapeutically effective administration of the recombinant, truncated human NEP of the invention typically occurs in doses ranging from 0.1 mg of the protein to kg of body weight to 25 mg/kg. In some embodiments, the therapeutically effective dose is 0.3, 1.0, 3, 5, 7.5, 10 and 25 mg/kg. Example 3 below provides an assay for the activity of an NEP, and therapeutically effective doses of other NEP or NEP homologs of the invention can be determined by measuring their activity relative to the activity of the recombinant, truncated human NEP of the invention and calculating the dose required to deliver an equivalent amount of activity. An amount effective to treat the disorders hereinbefore described depends upon such factors as the efficacy of the active compounds, the molecular weight of the NEP chosen, the nature and severity of the disorders being treated and the weight of the mammal. However, a unit dose will normally contain 0.01 to 200 mg, for example 20 to 100 mg, of the compound of the invention. “Unit dose” includes a discrete amount of the pharmaceutical composition comprising a predetermined amount of the active ingredient. In some embodiments, a dose of 1-200 mg of truncated NEP or an NEP homolog is injected as a single bolus in a human in need of treatment, including but not limited to a human with inflammatory bowel disease. In some embodiments, a dose of 20 to 100 mg is administered. In another embodiment, 1-200 mg of truncated NEP or NEP homolog is infused as a slow drip over the course of 1-4 hours. In some embodiments, a dose of 20 to 100 mg of truncated NEP or NEP homolog is infused intraveneously, e.g., as a slow drip over the course of 1 or more (e.g., 1-4) hours. In another embodiment, truncated NEP or NEP homolog is administered as a bolus of 1-200 mg, followed by an infusion of 1-200 mg over the course of one to six hours. In another embodiment, the dosing consists of slow infusion over the course of six to twelve hours. Doses for individual patients may be adjusted based on the weight or sex of the patient. Depending on the extent and severity of the disease, as many as 3 or four doses may be administered over a 3-4 week period for each disease incident.

A subject who has or is at risk of IBD is treated prior to the onset of one or more disease symptoms. Alternatively, the subject is treated concommittant to or after the onset of one or more disease symptoms. Therefore, the invention provides a method for preventing or reducing a symptom of inflammatory bowel disease in a mammalian subject, by identifying a mammalian subject at risk of inflammatory bowel disease and administering to the identified subject a NEP of the invention. A subject at risk of IBD is identified on the basis of family history, i.e., one or more parents, grandparents, siblings, issue, or other relatives have been diagnosed with IBD. Alternatively, a subject at risk of IBD is identified because the subject has a prior history of inflammatory bowel disease but is currently asymptomatic.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, domesticated animals, and animals used in agriculture.

As used herein, “administering” includes routes of administration which allow the compositions of the invention to perform their intended function, e.g., treating or preventing cardiac injury caused by hypoxia or ischemia. A variety of routes of administration are possible including, but not necessarily limited to parenteral (e.g., intravenous, intraarterial, intramuscular, subcutaneous injection), oral (e.g., dietary), topical, nasal, rectal, or via slow releasing microcarriers depending on the disease or condition to be treated. Oral, parenteral and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980)).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or diglycerides. Other parentally-administrable formulations that are useful include those, which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

An NEP of the invention can be delivered orally or via enema/suppository to treat inflammation of the bowel. For oral delivery, the present invention provides pharmaceutical compositions such that the NEP can pass into the small intestine without being destroyed by the harsh acidic environment of the stomach. Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

In one embodiment, the present invention provides NEP encapsulated in a polymer or other material that is resistant to acid hydrolysis or acid breakdown. In one embodiment, this formulation provides rapid release of NEP upon entry into the duodenum. Accordingly, the invention includes a composition containing an NEP and a pharmaceutically-acceptable acid-resistant (“enteric”) carrier. By acid-resistant is meant that the carrier or coating does not dissolve in an acidic environment. An acidic environment is characterized by a pH of less than 7. The acid-resistant carrier is resistant to acids at pH less than about 4.0. Preferably, the carrier does not dissolve in pH 2-3. Most preferably, it does not dissolve in pH of less than 2. In embodiments, the enteric coating is pH-sensitive. The coating dissolves after the pH is greater than 4.0. For example, the coating dissolves in a neutral environment as is encountered in the small intestine, and does not dissolve in an acidic environment as is encountered in the stomach. Alternatively, the enteric coating dissolves when exposed to specific metabolic event such as an encounter with a digestive enzyme that is found in the small intestine. For example, the coating is digested by a pancreatic enzyme such as trypsin, chymotrypsin, or a pancreatic lipase. Enteric coating materials are known in the art, e.g., malic acid-propane 1,2-diol. Cellulose derivatives, e.g., cellulose acetate phthalate or hydroxypropyl methylcellulose phthalate (HPMCP), are also useful in enteric acid-resistant coatings. Other suitable enteric coatings include cellulose acetate phthalate, polyvinyl acetate phthalate, methylcellulose, hydroxypropylmethylcellulose phthalate and anionic polymers of methacrylic acid and methyl methacrylate. Another suitable enteric coating is a water emulsion of ethylacrylate methylacrylic acid copolymer, or hydroxypropyl methyl cellulose acetate succinate (HPMAS). (See, e.g., U.S. Pat. Nos. 5,591,433, 5,750,104 and 4,079,125). An enteric coating is designed to resist solution in the stomach and to dissolve in the neutral or alkaline intestinal fluid. See also coatings described in Wilding et al., 1994, Targeting of drugs and vaccines to the gut, Pharmac. Ther. 62: 97-124, incorporated herein by reference. In another embodiment, lyophilized, particulate NEP mixed with bicarbonate (as buffer) is coated with Eudragit S100, L30D or L 100-44 according to the manufacturer's instructions (Rohm America).

In another embodiment, the formulations of the invention are those used successfully with lactase (see Langner, 1999, Enteric polymer coated capsule containing dried bacterial culture for supplying lactase, U.S. Pat. No. 6,008,027, incorporated herein by reference). In this embodiment, gelatin capsules are filled with 50-90% lyophilized NEP, the remaining capacity being filled with stabilizing dessicants such as silicon oxide, silicon dioxide or microcrystalline cellulose and bicarbonate buffer. The capsules are enterically coated with Eudragit polymer (Rohm America) or polyvinyl acetate phthalate (Sureteric, Merck Frosst) and vacuum dried prior to use. Similarly, diastase has been formulated with Eudragits RS 100 and cellulase acetate phthalate coatings for enteric use, and the present invention provides novel formulations that resemble these but contain NEP instead of diastase (see Vyas et al., 1991, Enteric spherules of diastase in enzyme preparations, J. Microencapsulation 8: 447-454, incorporated herein by reference).

To demonstrate that a formulation can increase NEP bioavailability in the small intestine, one uses any of the following tests. First, the ability of NEP activity to withstand 0.5-2 h of simulated gastric treatment (pepsin, in 0.1N HCI, pH 2) can be evaluated. If >10% activity can be reproducibly retained, the formulation is exposed to simulated conditions in the duodenum (pH 6.5 buffer containing trypsin, chymotrypsin and carboxypeptidase at a 1:100 molar ratio and elastase at a 1: 500 ratio to the NEP). In one embodiment, full release of NEP activity is achieved within 15 minutes. Formulations that satisfy the above criteria are tested in or more animal models of IBD, such as those described in Example 4 below.

Combination Therapies

The components of the combination therapies, as noted above, can be administered by the same route or by different routes.

“Combination therapy” also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Thus, the compounds of the invention and the other pharmacologically active agent may be administered to a patient simultaneously, sequentially or in combination. If administered sequentially, the time between administrations of each individual drug generally varies from 0.1 to about 48 hours. More preferably, the time between administrations varies from 4 hours and 24 hours. It will be appreciated that when using a combination of the invention, the compound of the invention and the other pharmacologically active agent may be in the same pharmaceutically acceptable carrier and therefore administered simultaneously. They may be in separate pharmaceutical carriers such as conventional oral dosage forms which are taken simultaneously. The term “combination” further refers to the case where the compounds are provided in separate dosage forms and are administered sequentially.

The following Examples are meant to be non-limiting and illustrate methods for making and using the invention.

### EXAMPLES

### Example 1

### Production of Human NEP

A. Cloning of Human NEP and Construction of Expression Vector

Human neutral endopeptidase is a 749 amino acid protein with an N-terminal transmembrane domain and a large extracellular domain that comprises an active protease domain. See Table 1. A truncation mutant lacking the transmembrane domain is generally more soluble than the full length protein and has more favorable physical characteristics for use as a therapeutic. To obtain the coding sequence of this domain, a LNCAP FGC human cell line was purchased from the American Type Culture Collection (ATCC), and cells were cultured in RPMI media in accordance with the specifications published in the ATCC bulletin. Whole cell RNA was extracted using Trizol, and approximately 200 micrograms (ug) of RNA were purified from an initial culture volume of 50 mL. RT PCR was used to amplify by PCR both full length and fragments of the human NEP gene. A PCR product encoding a polypeptide corresponding to amino acid residues 47-749 of SEQ ID NO: 2 (See, Genbank accession code: X07166; Swiss Prot ID P08473) was isolated and cloned into the Pichia expression vector pPICZα-A (Invitrogen catalog no. V195-20) using the XhoI and SacII restriction sites. The yeast Pichia pastoris expression system described herein is economical and can be used to produce large quantities of NEP. The design of the expression vector is such that the inserted coding sequence is placed in-frame with the Kex2 cleavage site, so that the coding sequence for the NEP is flush with the Kex2 cleavage site. Standard Recombinant DNA technology and oligonucleotide cassette mutagenesis was used to generate the coding sequence, as shown in Table 2.

TABLE 1

(SEQ ID NO: 1)

DNA SEQUENCE OF FULL LENGTH NEP:

| | |
|---|---|
| 1 | atggatataa ctgatatcaa cactccaaag ccaaagaaga aacagcgatg gactccactg |
| 61 | gagatcagcc tctcggtcct tgtcctgctc ctcaccatca tagctgtgac aatgatcgca |
| 121 | ctctatgcaa cctacgatga tggtatttgc aagtcatcag actgcataaa atcagctgct |
| 181 | cgactgatcc aaaacatgga tgccaccact gagccttgta cagacttttt caaatatgct |
| 241 | tgcgga9gct ggttgaaacg taatgtcatt cccgagacca gctcccgtta cggcaacttt |
| 301 | gacattttaa gagatgaact agaagtcgtt ttgaaagatg tccttcaaga acccaaaact |
| 361 | gaagatatag tagcagtgca gaaagcaaaa gcattgtaca ggtcttgtat aaatgaatct |
| 421 | gctattgata gcagaggtgg agaacctcta ctcaaactgt taccagacat atatgggtgg |
| 481 | ccagtagcaa cagaaaactg ggagcaaaaa tatggtgctt cttggacagc tgaaaaagct |
| 541 | attgcacaac tgaattctaa atatgggaaa aaagtcctta ttaatttgtt tgttggcact |
| 601 | gatgataaga attctgtgaa tcatgtaatt catattgacc aacctcgact tggcctccct |
| 661 | tctagagatt actatgaatg cactggaatc tataaagagg cttgtacagc atatgtggat |
| 721 | tttatgattt ctgtggccag attgattcgt caggaagaaa gattgcccat cgatgaaaac |
| 781 | cagcttgctt tggaaatgaa taaagttatg gaattggaaa aagaaattgc caatgctacg |
| 841 | gctaaacctg aagatcgaaa tgatccaatg cttctgtata acaagatgac attggcccag |
| 901 | atccaaaata acttttcact agagatcaat gggaagccat tcagctggtt gaatttcaca |
| 961 | aatgaaatca tgtcaactgt gaatattagt attacaaatg aggaagatgt ggttgtttat |
| 1021 | gctccagaat atttaaccaa acttaagccc attcttacca aatattctgc cagagatctt |
| 1081 | caaaatttaa tgtcctggag attcataatg gatcttgtaa gcagcctcag ccgaacctac |
| 1141 | aaggagtcca gaaatgcttt ccgcaaggcc ctttatggta caacctcaga aacagcaact |
| 1201 | tggagacgtt gtgcaaacta tgtcaatggg aatatggaaa atgctgtggg gaggctttat |
| 1261 | gtggaagcag catttgctgg agagagtaaa catgtggtcg aggatttgat tgcacagatc |
| 1321 | cgagaagttt ttattcagac tttagatgac ctcacttgga tggatgccga gacaaaaaag |
| 1381 | agagctgaag aaaaggcctt agcaattaaa gaaaggatcg gctatcctga tgacattgtt |
| 1441 | tcaaatgata acaaactgaa taatgagtac ctcgagttga actacaaaga agatgaatac |
| 1501 | ttcgagaaca taattcaaaa tttgaaattc agccaaagta aacaactgaa gaagctccga |
| 1561 | gaaaaggtgg acaaagatga gtggataagt ggagcagctg tagtcaatgc attttactct |
| 1621 | tcaggaagaa atcagatagt cttcccagcc ggcattctgc agcccccctt ctttagtgcc |
| 1681 | cagcagtcca actcattgaa ctatgggggc atcggcatgg tcataggaca cgaaatcacc |
| 1741 | catggcttcg atgacaatgg cagaaacttt aacaaagatg gagacctcgt tgactggtgg |
| 1801 | actcaacagt ctgcaagtaa ctttaaggag caatcccagt gcatggtgta tcagtatgga |
| 1861 | aacttttcct gggacctggc aggtggacag caccttaatg gaattaatac actgggagaa |
| 1921 | aacattgctg ataatggagg tcttggtcaa gcatacagag cctatcagaa ttatattaaa |
| 1981 | aagaatggcg aagaaaaatt acttcctgga cttgacctaa atcacaaaca actatttttc |
| 2041 | ttgaactttg cacaggtgtg gtgtggaacc tataggccag agtatgcggt taactccatt |
| 2101 | aaaacagatg tgcacagtcc aggcaatttc aggattattg ggactttgca gaactctgca |
| 2161 | gagttttcag aagcctttca ctgccgcaag aattcataca tgaatccaga aaagaagtgc |
| 2221 | cgggtttggt gatcttcaaa agaagcattg |

(SEQ ID NO: 2)

NEP amino acid sequence

| | |
|---|---|
| 1 | MDITDINTPK PKKKQRWTPL EISLSVLVLL LTIIAVTMIA LYATYDDGIC KSSDCIKSAA |
| 61 | RLIQNMDATT EPCTDFFKYA CGGWLKRNVI PETSSRYGNF DILRDELEVV LKDVLQEPKT |
| 121 | EDIVAVQKAK ALYRSCINES AIDSRGGEPL LKLLPDIYGW PVATENWEQK YGASWTAEKA |
| 181 | IAQLNSKYGK KVLINLFVGT DDKNSVNHVI HIDQPRLGLP SRDYYECTGI YKEACTAYVD |
| 241 | FMISVARLIR QEERLPIDEN QLALEMNKVN ELEKEIANAT AKPEDRNDPM LLYNKHTLAQ |
| 301 | IQNNFSLEIN GKPFSWLNFI NEIMSTVNIS ITNEEDVVVY APEYLTKLKP ILTKYSARDL |
| 361 | QNLMSWRFIM DLVSSLSRTY KESRNAFRKA LYGTTSETAT WRRCANYVNG NMENAVGRLY |
| 421 | VEAAFAGESK HVVEDLIAQI REVFIQTLDD LTWMDAETKK RAEEKALAIK ERIGYPDDIV |
| 481 | SNDNKLNNEY LELNYKEDEY FENIIQNLKF SQSKQLKKLR EKVDKDEWIS GAAVVNAFYS |
| 541 | SGRNQIVFPA GILQPPFFSA QQSNSLNYGG IGMVIGHEIT HGFDDNGRNF NKDGDLVDWW |
| 601 | TQQSASNFKE QSQCMVYQYG NFSWDLAGGQ HLNGINTLGE NIADNGGLGQ AYRAYQNYIK |
| 661 | KNGEEKLLPG LDLNHKQLFF LNFAQVWCGT YRPEYAVNSI KTDVHSPGNF RIIGTLQNSA |
| 721 | EFSEAFHCRK NSYMNPEKKC RVW* |

TABLE 2

(SEQ ID NO: 3)

| | |
|---|---|
| 1 | agatctaaca tccaaagacg aaaggttgaa tgaaaccttt ttgccatccg acatccacag |
| 61 | gtccattctc acacataagt gccaaacgca acaggagggg atacactagc agcagaccgt |
| 121 | tgcaaacgca ggacctccac tcctcttctc ctcaacaccc acttttgcca tcgaaaaacc |
| 181 | agcccagtta ttgggcttga ttggagctcg ctcattccaa ttccttctat taggctacta |
| 241 | acaccatgac tttattagcc tgtctatcct ggcccccctg gcgaggttca tgtttgttta |
| 301 | tttccgaatg caacaagctc cgcattacac ccgaacatca ctccagatga gggctttctg |
| 361 | agtgtggggt caaatagttt catgttcccc aaatggccca aaactgacag tttaaacgct |
| 421 | gtcttggaac ctaatatgac aaaagcgtga tctcatccaa gatgaactaa gtttggttcg |
| 481 | ttgaaatgct aacggccagt tggtcaaaaa gaaacttcca aaagtcggca taccgtttgt |
| 541 | cttgtttggt attgattgac gaatgctcaa aaataatctc attaatgctt agcgcagtct |
| 601 | ctctatcgct tctgaacccc ggtgcacctg tgccgaaacg caaatgggga aacacccgct |
| 661 | ttttggatga ttatgcattg tctccacatt gtatgcttcc aagattctgg tgggaatact |
| 721 | gctgatagcc taacgttcat gatcaaaatt taactgttct aacccctact tgacagcaat |
| 781 | atataaacag aaggaagctg ccctgtctta aacctttttt tttatcatca ttattagctt |
| 841 | actttcataa ttgcgactgg ttccaattga caagcttttg attttaacga cttttaacga |
| 901 | caacttgaga agatcaaaaa acaactaatt attcgaaacg atgagatttc cttcaatttt |
| 961 | tactgctgtt ttattcgcag catcctccgc attagctgct ccagtcaaca ctacaacaga |
| 1021 | agatgaaacg gcacaaattc cggctgaagc tgtcatcggt tactcagatt tagaagggga |
| 1081 | tttcgatgtt gctgttttgc cattttccaa cagcacaaat aacgggttat tgtttataaa |
| 1141 | tactactatt gccagcattg ctgctaaaga agaaggggta tctctcgaga aaagagatgg |
| 1201 | tatttgcaag tcatcagact gcataaaatc agctgctcga ctgatccaaa acatggatgc |
| 1261 | caccactgag ccttgtacag actttttcaa atatgcttgc ggaggctggt tgaaacgtaa |
| 1321 | tgtcattccc gagaccagct cccgttacgg caactttgac attttaagag atgaactaga |
| 1381 | agtcgttttg aaagatgtcc ttcaagaacc caaaactgaa gatatagtag cagtgcagaa |
| 1441 | agcaaaagca ttgtacaggt cttgtataaa tgaatctgct attgatagca gaggtggaga |
| 1501 | acctctactc aaactgttac cagacatata tgggtggcca gtagcaacag aaaactggga |
| 1561 | gcaaaaatat ggtgcttctt ggacagctga aaaagctatt gcacaactga attctaaata |
| 1621 | tgggaaaaaa gtccttatta atttgtttgt tggcactgat gataagaatt ctgtgaatca |
| 1681 | tgtaattcat attgaccaac ctcgacttgg cctcccttct agagattact atgaatgcac |
| 1741 | tggaatctat aaagaggctt gtacagcata tgtggatttt atgatttctg tggccagatt |
| 1801 | gattcgtcag gaagaaagat tgcccatcga tgaaaaccag cttgctttgg aaatgaataa |
| 1861 | agttatggaa ttggaaaaag aaattgccaa tgctacggct aaacctgaag atcgaaatga |
| 1921 | tccaatgctt ctgtataaca agatgacatt ggcccagatc caaaataact tttcactaga |
| 1981 | gatcaatggg aagccattca gctggttgaa tttcacaaat gaaatcatgt caactgtgaa |
| 2041 | tattagtatt acaaatgagg aagatgtggt tgtttatgct ccagaatatt taaccaaact |
| 2101 | taagcccatt cttaccaaat attctgccag agatcttcaa aatttaatgt cctggagatt |
| 2161 | cataatggat cttgtaagca gcctcagccg aacctacaag gagtccagaa atgctttccg |
| 2221 | caaggccctt tatggtacaa cctcagaaac agcaacttgg agacgttgtg caaactatgt |
| 2281 | caatgggaat atggaaaatg ctgtggggag gctttatgtg gaagcagcat ttgctggaga |
| 2341 | gagtaaacat gtggtcgagg atttgattgc acagatccga gaagttttta ttcagacttt |
| 2401 | agatgacctc acttggatgg atgccgagac aaaaaagaga gctgaagaaa aggccttagc |
| 2461 | aattaaagaa aggatcggct atcctgatga cattgtttca aatgataaca aactgaataa |
| 2521 | tgagtacctc gagttgaact acaaagaaga tgaatacttc gagaacataa ttcaaaattt |
| 2581 | gaaattcagc caaagtaaac aactgaagaa gctccgagaa aaggtggaca aagatgagtg |
| 2641 | gataagtgga gcagctgtag tcaatgcatt ttactcttca ggaagaaatc agatagtctt |
| 2701 | cccagccggc attctgcagc cccccttctt tagtgcccag cagtccaact cattgaacta |
| 2761 | tgggggcatc ggcatggtca taggacacqa aatcacccat ggcttcgatg acaatggcag |
| 2821 | aaactttaac aaagatggag acctcgttga ctggtggact caacagtctg caagtaactt |
| 2881 | taaggagcaa tcccagtgca tggtgtatca gtatggaaac ttttcctggg acctggcagg |
| 2941 | tggacagcac cttaatggaa ttaatacact gggagaaaac attgctgata atggaggtct |
| 3001 | tggtcaagca tacagagcct atcagaatta tattaaaaag aatggcgaag aaaaattact |
| 3061 | tcctggactt gacctaaatc acaaacaact atttttcttg aactttgcac aggtgtggtg |
| 3121 | tggaacctat aggccagagt atgcggttaa ctccattaaa acagatgtgc acagtccagg |
| 3181 | caatttcagg attattggga ctttgcagaa ctctgcagag ttttcagaag cctttcactg |
| 3241 | ccgcaagaat tcatacatga atccagaaaa gaagtgccgg gtttggtaat aaccgcggcg |
| 3301 | gccgccagct ttctagaaca aaaactcatc tcagaagagg atctgaatag cgccgtcgac |
| 3361 | catcatcatc atcatcattg agtttgtagc cttagacatg actgttcctc agttcaagtt |
| 3421 | gggcacttac gagaagaccg gtcttgctag attctaatca agaggatgtc agaatgccat |
| 3481 | ttgcctgaga gatgcaggct tcatttttga tactttttta tttgtaacct atatagtata |
| 3541 | ggattttttt tgtcattttg tttcttctcg tacgagcttg ctcctgatca gcctatctcg |
| 3601 | cagctgatga atatcttgtg gtaggggttt gggaaaatca ttcgagtttg atgtttttct |
| 3661 | tggtatttcc cactcctctt cagagtacag aagattaagt gagacCttcg tttgtgcgga |
| 3721 | tcccccacac accatagctt caaaatgttt ctactccttt tttactcttc cagattttct |
| 3781 | cggactccgc gcatcgccgt accacttcaa aacacccaag cacagcatac taaattttcc |
| 3841 | ctctttcttc ctctagggtg tcgttaatta cccgtactaa aggtttggaa aagaaaaaag |
| 3901 | agaccgcctc gtttcttttt cttcgtcgaa aaaggcaata aaaattttta tcacgtttct |
| 3961 | ttttcttgaa attttttttt ttagtttttt tctctttcag tgacctccat tgatatttaa |
| 4021 | gttaataaac ggtcttcaat ttctcaagtt tcagtttcat ttttcttgtt ctattacaac |
| 4081 | tttttttact tcttgttcat tagaaagaaa gcatagcaat ctaatctaag gggcggtgtt |
| 4141 | gacaattaat catcggcata gtatatcggc atagtataat acgacaaggt gaggaactaa |
| 4201 | accatggcca agttgaccag tgccgttccg gtgctcaccg cgcgcgacgt cgccggagcg |
| 4261 | gtcgagttct ggaccgaccg gctcgggttc tcccgggact tcgtggagga cgacttcgcc |
| 4321 | ggtgtggtcc gggacgacgt gaccctgttc atcagcgcgg tccaggacca ggtggtgccg |
| 4381 | gacaacaccc tggcctgggt gtgggtgcgc ggcctggacg agctgtacgc cgagtggtcg |
| 4441 | gaggtcgtgt ccacgaactt ccgggacgcc tccgggccgg ccatgaccga gatcggcgag |
| 4501 | cagccgtggg ggcgggagtt cgccctgcgc gacccggccg gcaactgcgt gcacttcgtg |
| 4561 | gccgaggagc aggactgaca cgtccgacgg cggcccacgg gtcccaggcc tcggagatcc |
| 4621 | gtcccccttt tcctttgtcg atatcatgta attagttatg tcacgcttac attcacgccc |
| 4681 | tccccccaca tccgctctaa ccgaaaagga aggagttaga caacctgaag tctaggtccc |
| 4741 | tatttatttt tttatagtta tgttagtatt aagaacgtta tttatatttc aaatttttct |
| 4801 | tttttttctg tacagacgcg tgtacgcatg taacattata ctgaaaacct tgcttgagaa |
| 4861 | ggttttggga cgctcgaagg ctttaatttg caagctggag accaacatgt gagcaaaagg |
| 4921 | ccagcaaaag gccaggaacc gtaaaaaggc cgcgttgctg gcgtttttcc ataggctccg |
| 4981 | cccccctgac gagcatcaca aaaatcgacg ctcaagtcag aggtggcgaa acccgacagg |
| 5041 | actataaaga taccaggcgt ttccccctgg aagctccctc gtgcgctctc ctgttccgac |
| 5101 | cctgccgctt accggatacc tgtccgcctt tctcccttcg ggaagcgtgg cgctttctca |
| 5161 | atgctcacgc tgtaggtatc tcagttcggt gtaggtcgtt cgctccaagc tgggctgtgt |
| 5221 | gcacgaaccc cccgttcagc ccgaccgctg cgccttatcc ggtaactatc gtcttgagtc |
| 5281 | caacccggta agacacgact tatcgccact ggcagcagcc actggtaaca ggattagcag |
| 5341 | agcgaggtat gtaggcggtg ctacagagtt cttgaagtgg tggcctaact acggctacac |
| 5401 | tagaaggaca gtatttggta tctgcgctct gctgaagcca gttaccttcg gaaaaagagt |
| 5461 | tggtagctct tgatccggca aacaaaccac cgctggtagc ggtggttttt ttgtttgcaa |
| 5521 | gcagcagatt acgcgcagaa aaaaaggatc tcaagaagat cctttgatct tttctacggg |
| 5581 | gtctgacgct cagtggaacg aaaactcacg ttaagggatt ttggtcatga gatc |

In this construct, the DNA sequence encoding the yeast alpha factor signal sequence corresponds to bases 941-1195 and is shown in bold. The DNA sequence encoding human NEP residues 47-749 (SWISSPROT accession code: P08473) corresponds to bases 1196-3292 and is underlined. The alpha-factor/NEP fusion protein translated from this construct is:

| (SEQ ID NO: 4) | |
|---|---|
| 1 | MRFPSIFTAV LFAASSALAA PVNTTTEDET AQIPAEAVIG YSDLEGDFDV AVLPFSNSTN |
| 61 | NGLLFINTTI ASIAAKEEGV SLEKRDGICK SSDCIKSAAR LIQNMDATTE PCTDFFKYAC |
| 121 | GGWLKRNVIP ETSSRYGNFD ILRDELEVVL KDVLQEPKTE DIVAVQKAKA LYRSCINESA |
| 181 | IDSRGGEPLL KLLPDIYGWP VATENWEQKY GASWTAEKAI AQLNSKYGKK VLINLFVGTD |
| 241 | DKNSVNHVIH IDQPRLGLPS RDYYECTGIY KEACTAYVDF MISVARLIRQ EERLPIDENQ |
| 301 | LALEMNKVME LEKEIANATA KPEDRNDPML LYNKMTLAQI QNNFSLEING KPFSWLNFTN |
| 361 | EIMSTVNISI TNEEDVVVYA PEYLTKLKPI LTKYSARDLQ NLMSWRFIMD LVSSLSRTYK |
| 421 | ESRNAFRKAL YGTTSETATW RRCANYVNGN MENAVGRLYV EAAFAGESKH VVEDLIAQIR |
| 481 | EVFIQTLDDL TWMDAETKKR AEEKALAIKE RIGYPDDIVS NDNKLNNEYL ELNYKEDEYF |
| 541 | ENIIQNLKFS QSKQLKKLRE KVDKDEWISG AAVVNAFYSS GRNQIVFPAG ILQPPFFSAQ |
| 601 | QSNSLNYGGI GMVIGHEITH GFDDNGRNFN KDGDLVDWWT QQSASNFKEQ SQCMVYQYGN |
| 661 | FSWDLAGGQH LNGINTLGEN IADNGGLGQA YRAYQNYIKK NGEEKLLPGL DLNHKQLFFL |
| 721 | NFAQVWCGTY RPEYAVNSIK TDVESPGNFR IIGTLQNSAE FSEAFHCRKN SYMNPEKKCR |
| 781 | VW** |

The underlined portion of the sequence above corresponds to NEP amino acids 47-749. The Kex2 protease cleaves between the alpha factor signal sequence and NEP, producing a mature form of the protease which is secreted into the media. Standard sequencing methods were used to verify that the desired construct, designated pPicZa-A-NEP, was obtained. The expressed protein is summarized as follows in Table 3:

TABLE 3

| | DNA | |
|---|---|---|
| NAME | SEQUENCE | PROTEIN SEQUENCE |
| Alpha factor | 941-1195 | MRFPSIFTAVLFAASSALAAPVNTTTEDETAQIPAEAVIG |
| signaling | | YSDLEGDFDVAVLPFSNSTNNGLLFINTTIASIAAKEEGV |
| peptide | | SLEKR Amino acids 1-85 of SEQ ID NO: 4 |
| Human NEP | 1196-3292 | Amino acids 47-749 of SWISSPROT ID: P08473 |

B. Screening for a High Level Expression Vector

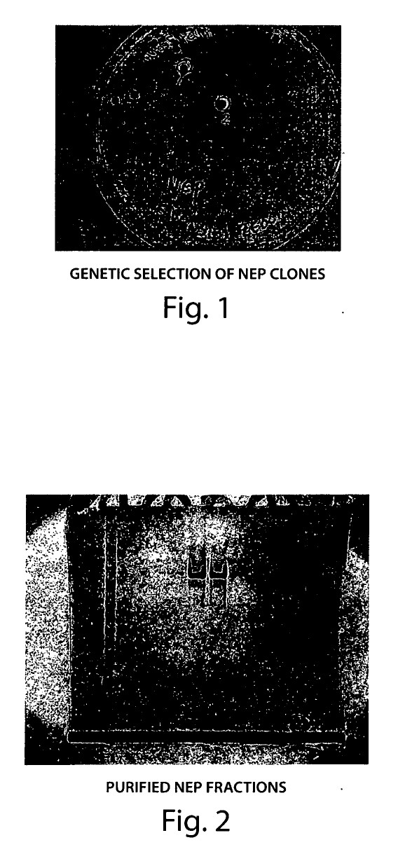

Previously published reports on the expression of NEP describe yields of approximately 1-10 mg/L (Gorman et al., J. Cell Biochem., 39:277-284 (1989), and Dale et al., Acta Cryst., D56:894-897 (2000)). These levels of production make cost prohibitive the production of therapeutic grade truncated NEP. The present invention provides an expression system that produces truncated NEP at substantially higher yeilds, and which are suitable for GMP production as a biological therapeutic. As outlined below, a genetic selection in the Pichia pastoris expression system was used to isolate a “jackpot” clone that contained multiple copies of DNA encoding truncated integrated into the Pichia chromosome(s) and expressed high levels of NEP. First, the pPicZα-A-NEP expression vector was linearized using the restriction enzyme SacI and then transformed into P. pastoris X33 cells by electroporation. Expression of a recombinant protein in Pichia is dependent on integration of the recombinant gene into the yeast genome. It has been shown that dramatically enhanced expression can be observed if multiple integration events occur during transformation. These “jackpot” clones (clones possessing multiple integrants) can be selected by using increasing amounts of the resistance marker zeocin. Approximately 10,000 colonies were plated onto standard YPDS plates containing zeocin, at concentrations of 100, 500, or 1000 ug/mL, in the growth media. As shown in FIG. 1, approximately 8 colonies were isolated which grew on plates containing zeocin at 500 ug/mL. Cells from each of these eight colonies were tested for expression in 25 mL baffled flasks. Starter cultures were grown in media containing 100 ug/mL of zeocin for two days to saturation, and then, glycerol stocks of each colony were stored at −80 degrees C. From the saturated starter culture, fresh cultures were grown to an OD=1-2 and then the pellets were harvested. The pellets were then re-suspended in YP media containing 0.5-10% methanol and grown anywhere from 1-12 days for production purposes. The jackpot clone produced approximately 40 mg/mL of truncated NEP when grown in standard baffled shake flasks. Typically, a >10 fold increase in expression is measured when scaling up from shake flasks to a fermentor due to increased aeration, precise control of nutrition, dissolved oxygen, pH, and other factors (Cregg and Higgins, Pichia Protocols, 1989). Therefore, the expression system and methods of the present invention are expected to produce truncated NEP at a level of at least 400 mg/L in fermentors. For a standard 5 Liter fermentation, this corresponds to the production of approximately 2 grams of NEP, which represents a forty-fold increase compared with previously published expression levels in a fermentation system.

### Example 2

### Purification of Truncated, Recombinant NEP

During the expression process using the pPicZα-A-NEP expression vector-containing P. pastoris cells of the invention, the NEP protein is secreted into the media. To purify the NEP protein in accordance with the methods of the invention, the bulk of contaminating proteins is removed by centrifugation of the media and removal of the cell pellet. After that, the protein is purified by slowly adding ammonium sulfate to the cell supernatant to a final concentration of about 60%. The precipitate that forms is removed by centrifugation; the NEP is in the soluble fraction, and the pellet containing the precipitate is discarded. This soluble fraction is then subjected to standard hydrophobic interaction chromatography. In one embodiment, this is accomplished using a column with a methyl or phenyl group coupled to a solid support such as Sepahrose. The soluble fraction is loaded in the presence of a high ionic strength buffer, such as, for example, 1.5 M ammonium sulfate containing 50 mM Tris, pH 7.4. The protein is then eluted from the resin in a column using a gradient of decreasing ammonium sulfate. Upon elution, the protein is >99% pure, as shown in FIG. 2; however, lipopolysaccharides were detected in this preparation, so in one embodiment, the purification method of the invention includes another purification step. The protein was dialyzed into 50 mM Tris, pH 7-8, loaded onto a UNO-Q6 high performance anion exchange column, and eluted using a gradient up to 100% 1.5 M NaCl, in Tris buffer, pH 7-8. The purity of the protein was examined an overloaded SDS page gel. After this step, the endoxtoxin levels were shown to be less than 10 EU/mL.

### Example 3

### NEP Enzyme Assay

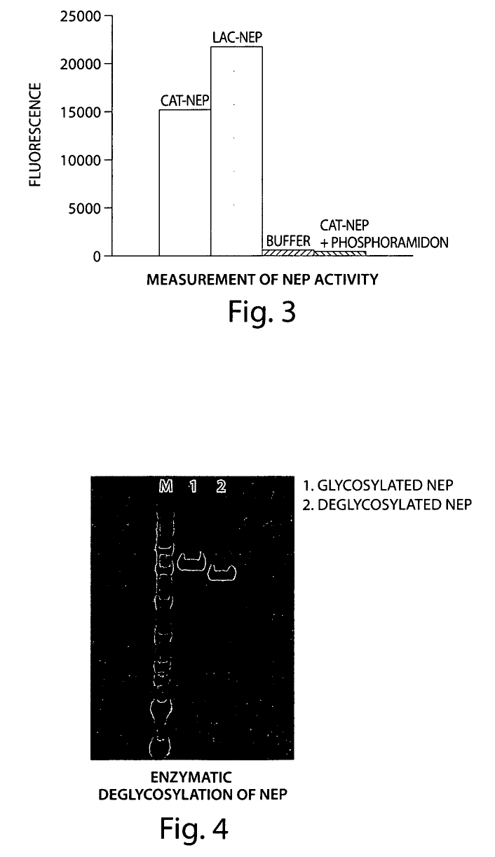

The activity of the NEP can be measured as follows. Succynl-Ala-Ala-Phe-aminomehtylcoumarin is a standard commercially available substrate (Sigma). Approximatley 10 nanograms of the NEP is incubated with the substrate at a concentration of 100 uM for 15 minutes at 37 degrees. At that time, phosphoramidon, an inhibitor to NEP, is added to the mixture in excess to terminate the reaction. At this point, aminopeptidase M (Sigma) which degrades amino terminus containing peptides, frees the fluorescent AMC leaving group only in the substrates internally hydrolyzed by NEP. The reaction is further incubated for 15 minutes at 37 degrees C., and then fluorescence of the AMC group is measured using a standard plate reader, such as a Spectramax Gemini (Molecular Devices, Inc.). FIG. 3 shows the results of this assay from a typical test. In the figure, “CAT-NEP” is a recombinant, truncated NEP prepared in accordance with the methods of the invention; “LAC-NEP” is NEP cloned, expressed, and purified from the benign intestinal bacteria; Lactococcus Lactis. “Buffer” is a negative control sample not containing any added NEP.

### Example 4

### Enzymatic and Engineered Deglycosylation and of NEP

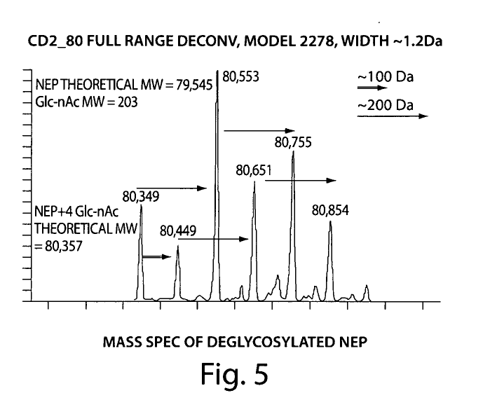

Expression of NEP in the methlyltropic yeast pichia pastoris results in the in a final expessed protein product that has a non-native, high mannose containing N-linked glycosylation pattern, which is typical of proteins expressed in yeast. These post-translational modifications can be highly immunogenic and cause rapid clearance in mammals. In this example we describe the enzymatic removal of such N-linked sugars with the enzyme Endoglycosidase F1, which leaves a single glc-nac hexose modification on each modified Asparagine residue. Upon purification of NEP as shown in Example 2, the protein is treated with EndoF1 as follows: (1) a high quality source of Endo F1 (sourced from either Calbiochem or Q&A Bio) is mixed with recombinant truncated NEP at a ratio of 0.01-0.10% (w/w) EndoF1:NEP (final) in either 50 mM Sodium Acetate buffer, pH 5.5 or Phosphate Buffered Saline. For example, 250 milligrams of NEP (at a concentration of 1-10 mg/ml) is incubated with 25-250 micrograms of EndoF1 for 2 hours at room temperature or overnight at 4 degrees Celsius. Upon enzymatic deglycosylation of NEP, the protein is loaded onto a S12 cation exhange column (BIO RAD) at pH 5 in 50 mM Sodium Acetate. Generally, NEP is bound onto the column at this pH. A gradient is run from pH 5 to 5.5 (all in 50 mM NaOAc) and NEP elutes at approximately pH 5.25. Most contaminating proteins do not elute during this process, resulting in a very efficient purification step. Catalytically inactive NEP is also separated, resulting in a process that allows one to isolate NEP with high specific activity. The Endo F1 treated NEP runs at approximately 10 KDa lower molecular weight on a SDS page polyacrylamide gel (FIG. 4). Electrospray mass spectrometry of fully glycosylated NEP shows a molecular weight range in an envelope of 87-92 kDa. Electrospray mass spectrometry of EndoF1 treated NEP shows a molecular weight of 80349 kDa, which corresponds to the calculated molecular weight of NEP plus four additional glc-nac residues, suggesting that there are at least four N-linked glycosylation sites on NEP which are fully occupied (FIG. 5). There are two additional peaks of 80553 kDa (corresponding to the molecular weight of NEP plus five glc-nacs), and 80755 kDa (corresponding to the molecular weight of NEP plus 6 glc-nacs) which suggests that there are two additional N-linked glycosylation sites in NEP which are partially occupied (FIG. 5). These results are consistent with the primary amino acid sequence of recombinant truncated NEP, which has six putative N-linked glycosylation sites (the N-linked Glycosylation signature corresponds to the tripeptide amino acid pattern N—X—S/T, where N is Asparagine, X is any amino acid, S is Serine, and T is Threonine), as shown below. The sites of N-linked glycosylation in recombinant truncated NEP are N144, N284, N310, N324, N334, and N627 (NEP encoding peptide is underlined and the N-linked glycosylation sites are shown in bold):

| (SEQ ID NO: 4) | |
|---|---|
| 1 | MRFPSIFTAV LFAASSALAA PVNTTTEDET AQIPAEAVIG YSDLEGDFDV AVLPFSNSTN |
| 61 | NGLLFINTTI ASIAAKEEGV SLEKRDGICK SSDCIKSAAR LIQNMDATTE PCTDFFKYAC |
| 121 | GGWLKRNVIP ETSSRYGNFD ILRDELEVVL KDVLQEPKTE DIVAVQKAKA LYRSCINESA |
| 181 | IDSRGGEPLL KLLPDIYGWP VATENWEQKY GASWTAEKAI AQLNSKYGKK VLINLFVGTD |
| 241 | DKNSVNHVIH IDQPRLGLPS RDYYECTGIY KEACTAYVDF MISVARLIRQ EERLPIDENQ |
| 301 | LALEMNKVME LEKEIANATA KPEDRNDPML LYNKMTLAQI QNNFSLEING KPFSWLNFTN |
| 361 | EIMSTVNISI TNEEDVVVYA PEYLTKLKPI LTKYSARDLQ NLMSWRFIMD LVSSLSRTYK |
| 421 | ESRNAFRKAL YGTTSETATW RRCANYVNGN MENAVGRLYV EAAFAGESKH VVEDLIAQIR |
| 481 | EVFIQTLDDL TWMDAETKKR AEEKALAIKE RIGYPDDIVS NDNKLNNEYL ELNYKEDEYF |
| 541 | ENIIQNLKFS QSKQLKKLRE KVDKDEWISG AAVVNAFYSS GRNQIVFPAG ILQPPFFSAQ |
| 601 | QSNSLNYGGI GMVIGHEITH GFDDNGRNFN KDGDLVDWWT QQSASNFKEQ SQCMVYQYGN |
| 661 | FSWDLAGGQH LNGINTLGEN IADNGGLGQA YRAYQNYIKK NGEEKLLPGL DLNHKQLFFL |
| 721 | NFAQVWCGTY RPEYAVNSIK TDVESPGNFR IIGTLQNSAE FSEAFHCRKN SYMNPEKKCR |
| 781 | VW** |

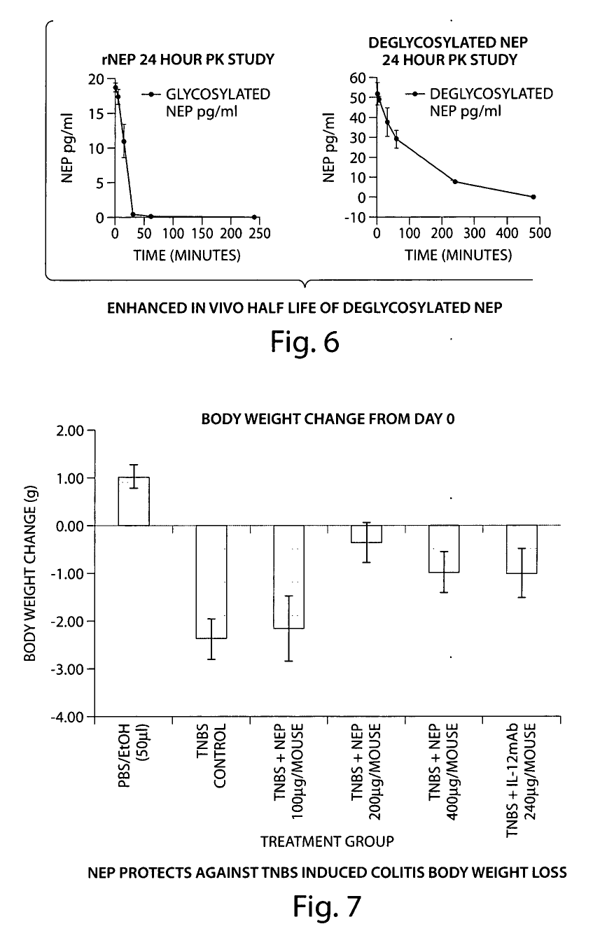

Recombinant, truncated fully glycosylated or enzymatically deglycosylated NEP was tested for increased in-vivo half life in mice by the following experiment: 100 μl of NEP, (3 mg/ml) was injected into the tail vein of male Swiss Webster mice. Blood was drawn at 1, 5, 15, 30, 60 min, 4 hr, 12 hr, and 24 hr (3 mice sacrificed per time point). The mice were exsanguinated and the plasma isolated (approximately 500 μl). The presence of recombinant, truncated NEP was tested by the use of the following ELISA. An anti human NEP antibody (R&D Systems) was coupled to biotin using standard chemistry (Pierce) and bound onto a strepdavidin (Pierce) coated maxysorp plate. 100 μl of NEP containing plasma, where each well represents a time point in the 24 hour PK study (done in triplicate), was loaded per well onto the plate and allowed to incubate, mixing, for one hour at room temperature. The plate was washed 3 times with 200 μl of 1× PBS plus 0.01% Tween 20. An anti human NEP-HRP conjugate was synthesized using standard coupling chemistry (Pierce, R&D Systems), purified, added to the plate, mixed, and allowed to incubate with the sample for 30 minutes, shaking at room temperature. The ELISA was then developed using a standard TMB substrate solution (Pierce). Fully glycosylated recombinant, truncated NEP has an in vivo half life of approximately 10 minutes and enzymatically deglycosylated recombinant, truncated NEP has an in vivo half life of >100 minutes (FIG. 6). Thus, enzymatic deglycosylation of NEP results in a form of the protein that has >10 fold increased in vivo half over the material that is naturally produced in pichia pastoris.

### Example 5

A variant of NEP which has site-directed N to Q mutations in each of the 6 known N-linked glycosylation sites has advantages over the naturally produced material in that a) the protein does not have to be enzymatically deglycosylated, which eliminates a costly manufacturing step, b) the potentially immunogenic or destabilizing residual glc-nac on each of the 6 Asparagine residuces is avoided, and c) the final, purified material is generally free of any heterogeneous glycoforms. This construct, prepared as in example 1, is enoded by the following DNA sequence where the genetic mutations encoding the asparagine to glutamine variants are in bold. The mutants correspond to the NEP primary amino acid sequence as follows: N144Q, N284Q, N310Q, N324Q, N334Q, and N627Q and the resultant construct is named NEP(N144Q/N284Q/N310Q/N324Q/N334Q/N627Q):

| (SEQ ID NO: 5) | |
|---|---|
| 1 | agatctaaca tccaaagacg aaaggttgaa tgaaaccttt ttgccatccg acatccacag |
| 61 | gtccattctc acacataagt gccaaacyca acaggagggg atacactagc agcagaccgt |
| 121 | tgcaaacgca ggacctccac tcctcttctc ctcaacaccc acttttgcca tcgaaaaacc |
| 181 | agcccagtta ttgggcttga ttggagctcg ctcattccaa ttccttctat taggctacta |
| 241 | acaccatgac tttattagcc tgtctatcct ggcccccctg gcgaggttca tgtttgttta |
| 301 | tttccgaatg caacaagctc cgcattacac ccgaacatca ctccagatga gggctttctg |
| 361 | agtgtggggt caaatagttt catgttcccc aaatggccca aaactgacag tttaaacgct |
| 421 | gtcttggaac ctaatatgac aaaagcgtga tctcatccaa gatgaactaa gtttggttcg |
| 481 | ttgaaatgct aacggccagt tggtcaaaaa gaaacttcca aaagtcggca taccgtttgt |
| 541 | cttgtttggt attgattgac gaatgctcaa aaataatctc attaatgctt agcgcagtct |
| 601 | ctctatcgct tctgaacccc ggtgcacctg tgccgaaacg caaatgggga aacacccgct |
| 661 | ttttggatga ttatgcattg tctccacact gtatgcttcc aagattctgg tgggaatact |
| 721 | gctgatagcc taacgttcat gatcaaaatt taactgtcct aacccctact tgacagcaat |
| 781 | atataaacag aaggaagctg ccctgtctta aacctttttt tttatcatca ttattagctt |
| 841 | actttcataa ttgcgactgg ttccaattga caagcttttg attttaacga cttttaacga |
| 901 | caacttgaga agatcaaaaa acaactaatt attcgaaacg atgagatttc cttcaatttt |
| 961 | tactgctgtt ttattcgcag catcctccgc attagctgct ccagtcaaca ctacaacaga |
| 1021 | agatgaaacg gcacaaattc cggctgaagc tgtcatcggt tactcagatt tagaagggga |
| 1081 | tttcgatgtt gctgttttgc cattttccaa cagcacaaat aacgggttat tgtttataaa |
| 1141 | tactactatt gccagcattg ctgctaaaga agaaggggta tctctcgaga aaagagatgg |
| 1201 | tatttgcaag tcatcagact gcataaaatc agctgctcga ctgatccaaa acatggatgc |
| 1261 | caccactgag ccttgtacag actttttcaa atatgcttgc ggaggctggt tgaaacgtaa |
| 1321 | tgtcattccc gagaccagct cccgttacgg caactttgac attttaagag atgaactaga |
| 1381 | agtcgttttg aaagatgtcc ttcaagaacc caaaactgaa gatatagtag cagtgcagaa |
| 1441 | agcaaaagca ttgtacaggt cttgtataCA Gaatctgct attgatagca gaggtggaga |
| 1501 | acctctactc aaactgttac cagacatata tgggtggcca gtagcaacag aaaactggga |
| 1561 | gcaaaaatat ggtgcttctt ggacagctga aaaagctatt gcacaactga attctaaata |
| 1621 | tgggaaaaaa gtccttatta atttgtttgt tggcactgat gataagaatt ctgtgaatca |
| 1681 | tgtaattcat attgaccaac ctcgacttgg cctcccttct agagattact atgaatgcac |
| 1741 | tggaatctat aaagaggctt gtacagcata tgtggatttt atgatttctg tggccagatt |
| 1801 | gattcgtcag gaagaaagat tgcccatcga tgaaaaccag cttgctttgg aaatgaataa |
| 1861 | agttatggaa ttggaaaaag aaattgccCA Ggctacggct aaacctgaag atcgaaatga |
| 1921 | tccaatgctt ctgtataaca agatgacatt ggcccagatc caaaatCAGt tttcactaga |
| 1981 | gatcaatggg aagccattca gctggttgCA Gttcacaaat gaaatcatgt caactgtgCA |
| 2041 | Gattagtatt acaaatgagg aagatgtggt tgtttatgct ccagaatatt taaccaaact |
| 2101 | taagcccatt cttaccaaat attctgccag agatcttcaa aatttaatgt cctggagatt |
| 2161 | cataatggat cttgtaagca gcctcagccg aacctacaag gagtccagaa atgctttccg |
| 2221 | caaggccctt tatggtacaa cctcagaaac agcaacttgg agacgttgtg caaactatgt |
| 2281 | caatgggaat atggaaaatg ctgtggggag gctttatgtg gaagcagcat ttgctggaga |
| 2341 | gagtaaacat gtggtcgagg atttgattgc acagatccga gaagttttta ttcagacttt |
| 2401 | agatgacctc acttggatgg atgccgagac aaaaaagaga gctgaagaaa aggccttagc |
| 2461 | aattaaagaa aggatcggct atcctgatga cattgtttca aatgataaca aactgaataa |
| 2621 | tgagtacctc gagttgaact acaaagaaga tgaatacttc gagaacataa ttcaaaattt |
| 2581 | gaaattcagc caaagtaaac aactgaagaa gctccgagaa aaggtggaca aagatgagtg |
| 2641 | gataagtgga gcagctgtag tcaatgcatt ttactcttca ggaagaaatc agatagtctt |
| 2701 | cccagccggc attctgcagc cccccttctt tagtgcccag cagtccaact cattgaacta |
| 2761 | tgggggcatc ggcatggtca taggacacga aatcacccat ggcttcgatg acaatggcag |
| 2821 | aaactttaac aaagatggag acctcgttga ctggtggact caacagtctg caagtaactt |
| 2881 | taaggagcaa tcccagtgca tggtgtatca gtatggacaa ttttcctggg acctggcagg |
| 2941 | tggacagcac cttaatggaa ttaatacact gggagaaaac attgctgata atggaggtct |
| 3001 | tggtcaagca tacagagcct atcagaatta tattaaaaag aatggcgaag aaaaattact |
| 3061 | tcctggactt gacctaaatc acaaacaact atttttcttg aactttgcac aggtgtggtg |
| 3121 | tggaacctat aggccagagt atgcggttaa ctccattaaa acagatgtgc acagtccagg |
| 3181 | caatttcagg attattggga ctttgcagaa ctctgcagag ttttcagaag cctttcactg |
| 3241 | ccgcaagaat tcatacatga atccagaaaa gaagtgccgg gtttggtaat aaccgcggcg |
| 3301 | gccgccagct ttctagaaca aaaactcatc tcagaagagg atctgaatag cgccgtcgac |
| 3361 | catcatcatc atcatcattg agtttgtagc cttagacatg actgttcctc agttcaagtt |
| 3421 | gggcacttac gagaagaccg gtcttgctag attctaatca agaggatgtc agaatgccat |
| 3481 | ttgcctgaga gatgcaggct tcatttttga tactttttta tttgtaacct atatagtata |
| 3541 | ggattttttt tgtcattttg tttcttctcg tacgagcttg ctcctgatca gcctatctcg |
| 3601 | cagctgatga atatcttgtg gtaggggttt gggaaaatca ttcgagtttg atgtttttct |
| 3661 | tggtatttcc cactcctctt cagagtacag aagattaagt gagaccttcg tttgtgcgga |
| 3721 | tcccccacac accatagctt caaaatgttt ctactccttt tttactcttc cagattttct |
| 3781 | cggactccgc gcatcgccgt accacttcaa aacacccaag cacagcatac taaattttcc |
| 3841 | ctctttcttc ctctagggtg tcgttaatta cccgtactaa aggtttggaa aagaaaaaag |
| 3901 | agaccgcctc gtttcttttt cttcgtcgaa aaaggcaata aaaattttta tcacgtttct |
| 3961 | ttttcttgaa attttttttt ttagtttttt tctctttcag tgacctccat tgatatttaa |
| 4021 | gttaataaac ggtcttcaat ttctcaagtt tcagtttcat ttttcttgtt ctattacaac |
| 4081 | tttttttact tcttgttcat tagaaagaaa gcatagcaat ctaatctaag gggcggtgtt |
| 4141 | gacaattaat catcggcata gtatatcggc atagtataat acgacaaggt gaggaactaa |
| 4201 | accatggcca agttgaccag tgccgttccg gtgctcaccg cgcgcgacgt cgccggagcg |
| 4261 | gtcgagttct ggaccgaccg gctcgggttc tcccgggact tcgtggagga cgacttcgcc |
| 4321 | ggtgtggtcc gggacgacgt gaccctgttc atcagcgcgg tccaggacca ggtggtgccg |
| 4381 | gacaacaccc tggcctgggt gtgggtgcgc ggcctggacg agctgtacgc cgagtggtcg |
| 4441 | gaggtcgtgt ccacgaactt ccgggacgcc tccgggccgg ccatgaccga gatcggcgag |
| 4501 | cagccgtggg ggcgggagtt cgccctgcgc gacccggccg gcaactgcgt gcacttcgtg |
| 4561 | gccgaggagc aggactgaca cgtccgacgg cggcccacgg gtcccaggcc tcggagatcc |
| 4621 | gtcccccttt tcctttgtcg atatcatgta attagttatg tcacgcttac attcacgccc |
| 4681 | tccccccaca tccgctctaa ccgaaaagga aggagttaga caacctgaag tctaggtccc |
| 4741 | tatttatttt tttatagtta tgttagtatt aagaacgtta tttatatttc aaatttttct |
| 4801 | tttttttctg tacagacgcg tgtacgcatg taacattata ctgaaaacct tgcttgagaa |
| 4861 | ggttttggga cgctcgaagg ctttaatttg caagctggag accaacatgt gagcaaaagg |
| 4921 | ccagcaaaag gccaggaacc gtaaaaaggc cgcgttgctg gcgtttttcc ataggctccg |
| 4981 | cccccctgac gagcatcaca aaaatcgacg ctcaagtcag aggtggcgaa acccgacagg |
| 5041 | actataaaga taccaggcgt ttccccctgg aagctccctc gtgcgctctc ctgttccgac |
| 5101 | cctgccgctt accggatacc tgtccgcctt tctcccttcg ggaagcgtgg cgctttctca |
| 5161 | atgctcacgc tgtaggtatc tcagttcggt gtaggtcgtt cgctccaagc tgggctgtgt |
| 5221 | gcacgaaccc cccgttcagc ccgaccgctg cgccttatcc ggtaactatc gtcttgagtc |
| 5281 | caacccggta agacacgact tatcgccact ggcagcagcc actggtaaca ggattagcag |
| 5341 | agcgaggtat gtaggcggtg ctacagagtt cttgaagtgg tggcctaact acggctacac |
| 5401 | tagaaggaca gtatttggta tctgcgctct gctgaagcca gttaccttcg gaaaaagagt |
| 5461 | tggtagctct tgatccggca aacaaaccac cgctggtagc ggtggttttt ttgtttgcaa |
| 5521 | gcagcagatt acgcgcagaa aaaaaggatc tcaagaagat cctttgatct tttctacggg |
| 5581 | gtctgacgct cagtggaacg aaaactcacg ttaagggatt ttggtcatga gatc |

The underlined portion of the sequence above corresponds to NEP amino acids 47-749. The Kex2 protease cleaves between the alpha factor signal sequence (shown in bold) and NEP, producing a mature form of the protease which is secreted into the media. Standard sequencing methods were used to verify that the desired construct, designated pPicZa-A-NEP, was obtained. The expressed protein is summarized as follows:

| | DNA | |
|---|---|---|
| NAME | SEQUENCE | PROTEIN SEQUENCE |
| Alpha factor | 941-1195 | MRFPSIFTAVLFAASSALAAPVNTTTEDETAQIPAEAVIG |
| signaling | | YSDLEGDFDVAVLPFSNSTNNGLLFINTTIASIAAKEEGV |
| peptide | | SLEKR |
| Human NEP | 1196-3292 | Amino acids 47-749 of SWISSPROT ID: P08473 |
| | | N144Q/N284Q/N310Q/N324Q/N334Q/N627Q |

In order to prevent rapid in-vivo clearance of NEP and to maximize its anti-inflammatory activity, the protein may be fused to peptides which bind to long-lived serum proteins such as serum albumin, fibrinogen, and antibodies, or other proteins present in the vasculature or serum (e.g., cell surface proteins of endothelial cells, and the neonatal Fc receptor (FcRn)). These NEP variants generally exhibit an increased circulating half life, compared with wild type NEP, due to their binding to long lived serum proteins or cell surface proteins, and will therefore increase in-vivo exposure of NEP to its natural substrates. This increased exposure of NEP to its substrates increases the therapeutic effect of the protein. For example, the following construct encodes a fusion protein between a peptide that binds to both human and mouse serum albumin (DRLIEDICLPRWGCLWEDDGS) (SEQ ID NO: 6). This peptide was fused to an antibody Fab Fragment (Dennis et al, 2002) and increased the serum half life of the protein 25-50 fold when tested in both mice and rabbits.

| (SEQ ID NO: 7) | |
|---|---|
| 1 | agatctaaca tccaaagacg aaaggttgaa tgaaaccttt ttgccatccg acatccacag |
| 61 | gtccattctc acacataagt gccaaacgca acaggagggg atacactagc agcagaccgt |
| 121 | tgcaaacgca ggacctccac tcctcttctc ctcaacaccc acttttgcca tcgaaaaacc |
| 181 | agcccagtta ttgggcttga ttggagctcg ctcattccaa ttccttctat taggctacta |
| 241 | acaccatgac tttattagcc tgtctatcct ggcccccctg gcgaggttca tgtttgttta |
| 301 | tttccgaatg caacaagctc cgcattacac ccgaacatca ctccagatga gggctttctg |
| 361 | agtgtggggt caaatagttt catgttcccc aaatggccca aaactgacag tttaaacgct |
| 421 | gtcttggaac ctaatatgac aaaagcgtga tctcatccaa gatgaactaa gtttggttcg |
| 481 | ttgaaatgct aacggccagt tggtcaaaaa gaaacttcca aaagtcggca taccgtttgt |
| 541 | cttgtttggt attgattgac gaatgctcaa aaataatctc attaatgctt agcgcagtct |
| 601 | ctctatcgct tctgaacccc ggtgcacctg tgccgaaacg caaatgggga aacacccgct |
| 661 | ttttggatga ttatgcattg tctccacatt gtatgcttcc aagattctgg tgggaatact |
| 721 | gctgatagcc taacgttcat gatcaaaatt taactgttct aacccctact tgacagcaat |
| 781 | atataaacag aaggaagctg ccctgtctta aacctttttt tttatcatca ttattagctt |
| 841 | actttcataa ttgcgactgg ttccaattga caagcttttg attttaacga cttttaacga |
| 901 | caacttgaga agatcaaaaa acaactaatt attcgaaacg atgagatttc cttcaatttt |
| 961 | tactgctgtt ttattcgcag catcctccgc attagctgct ccagtcaaca ctacaacaga |
| 1021 | agatgaaacg gcacaaattc cggctgaagc tgtcatcggt tactcagatt tagaagggga |
| 1081 | tttcgatgtt gctgttttgc cattttccaa cagcacaaat aacgggttat tgtttataaa |
| 1141 | tactactatt gccagcattg ctgctaaaga agaaggggta tctctcgaga aaagagaccg |
| 1201 | cctgatcgag gatatctgcc tgccccggtg gggctgcctg tgggaggatg atggtagtga |
| 1261 | tggtatttgc aagtcatcag actgcataaa atcagctgct cgactgatcc aaaacatgga |
| 1321 | tgccaccact gagccttgta cagacttttt caaatatgct tgcggaggct ggttgaaacg |
| 1381 | taatgtcatt cccgagacca gctcccgtta cggcaacttt gacattttaa gagatgaact |
| 1441 | agaagtcgtt ttgaaagatg tccttcaaga acccaaaact gaagatatag tagcagtgca |
| 1501 | gaaagcaaaa gcattgtaca ggtcttgtat aaatgaatct gctattgata gcagaggtgg |
| 1561 | agaacctcta ctcaaactgt taccagacat atatgggtgg ccagtagcaa cagaaaactg |
| 1621 | ggagcaaaaa tatggtgctt cttggacagc tgaaaaagct attgcacaac tgaattctaa |
| 1681 | atatgggaaa aaagtcctta ttaatttgtt tgttggcact gatgataaga attctgtgaa |
| 1741 | tcatgtaatt catattgacc aacctcgact tggcctccct tctagagatt actatgaatg |
| 1801 | cactggaatc tataaagagg cttgtacagc atatgtggat tttatgattt ctgtggccag |
| 1861 | attgattcgt caggaagaaa gattgcccat cgatgaaaac cagcttgctt tggaaatgaa |
| 1921 | taaagttatg gaattggaaa aagaaattgc caatgctacg gctaaacctg aagatcgaaa |
| 1981 | tgatccaatg cttctgtata acaagatgac attggcccag atccaaaata acttttcact |
| 2041 | agagatcaat gggaagccat tcagctggtt gaatttcaca aatgaaatca tgtcaactgt |
| 2101 | gaatattagt attacaaatg aggaagatgt ggttgtttat gctccagaat atttaaccaa |
| 2161 | acttaagccc attcttacca aatattctgc cagagatctt caaaatttaa tgtcctggag |
| 2221 | attcataatg gatcttgtaa gcagcctcag ccgaacctac aaggagtcca gaaatgcttt |
| 2281 | ccgcaaggcc ctttatggta caacctcaga aacagcaact tggagacgtt gtgcaaacta |
| 2341 | tgtcaatggg aatatggaaa atgctgtggg gaggctttat gtggaagcag catttgctgg |
| 2401 | agagagtaaa catgtggtcg aggatttgat tgcacagatc cgagaagttt ttattcagac |
| 2461 | tttagatgac ctcacttgga tggatgccga gacaaaaaag agagctgaag aaaaggcctt |
| 2521 | agcaattaaa gaaaggatcg gctatcctga tgacattgtt tcaaatgata acaaactgaa |
| 2581 | taatgagtac ctcgagttga actacaaaga agatgaatac ttcgagaaca taattcaaaa |
| 2641 | tttgaaattc agccaaagta aacaactgaa gaagctccga gaaaaggtgg acaaagatga |
| 2701 | gtggataagt ggagcagctg tagtcaatgc attttactct tcaggaagaa atcagatagt |
| 2761 | cttcccagcc ggcattctgc agcccccctt ctttagtgcc cagcagtcca actcattgaa |
| 2821 | ctatgggggc atcggcatgg tcataggaca cgaaatcacc catggcttcg atgacaatgg |
| 2881 | cagaaacttt aacaaagatg gagacctcgt tgactggtgg actcaacagt ctgcaagtaa |
| 2941 | ctttaaggag caatcccagt gcatggtgta tcagtatgga aacttttcct gggacctggc |
| 3001 | aggtggacag caccttaatg gaattaatac actgggagaa aacattgctg ataatggagg |
| 3061 | tcttggtcaa gcatacagag cctatcagaa ttatattaaa aagaatggcg aagaaaaatt |
| 3121 | acttcctgga cttgacctaa atcacaaaca actatttttc ttgaactttg cacaggtgtg |
| 3181 | gtgtggaacc tataggccag agtatgcggt taactccatt aaaacagatg tgcacagtcc |
| 3241 | aggcaatttc aggattattg ggactttgca gaactctgca gagttttcag aagcctttca |
| 3301 | ctgccgcaag aattcataca tgaatccaga aaagaagtgc cgggtttggt aataaccgcg |
| 3361 | gcggccgcca gctctctaga acaaaaactc atctcagaag aggatctgaa tagcgccgtc |
| 3421 | gaccatcatc atcatcatca ttgagtttgt agccttagac atgactgttc ctcagttcaa |
| 3481 | gttgggcact tacgagaaga ccggtcttgc tagattctaa tcaagaggat gtcagaatgc |
| 3541 | catttgcctg agagatgcag gcttcatttt tgatactttt ttatttgtaa cctatatagt |
| 3601 | ataggatttt ttttgtcatt ttgtttcttc tcgtacgagc ttgctcctga tcagcctatc |
| 3661 | tcgcagctga tgaatatctt gtggtagggg tttgggaaaa tcattcgagt ttgatgtttt |
| 3721 | tcttggtatt tcccactcct cttcagagta cagaagatta agtgagacct tcgtttgtgc |
| 3781 | ggatccccca cacaccatag cttcaaaatg tttctactcc ttttttactc ttccagattt |
| 3841 | tcccggactc cgcgcatcgc cgtaccactt caaaacaccc aagcacagca tactaaattt |
| 3901 | tccctctttc ttcctctagg gtgtcgttaa ttacccgtac taaaggtttg gaaaagaaaa |
| 3961 | aagagaccgc ctcgtttctt tttcttcgtc gaaaaaggca ataaaaattt ttatcacgtt |
| 4021 | tctttttctt gaaatttttt tttttagttt ttttctcttt cagtgacctc cattgatatt |
| 4081 | taagttaata aacggtcttc aatttctcaa gtttcagttt catttttctt gttctattac |
| 4141 | aacttttttt acttcttgtt cattagaaag aaagcatagc aatctaatct aaggggcggt |
| 4201 | gttgacaatt aatcatcggc atagtatatc ggcatagtat aatacgacaa ggtgaggaac |
| 4261 | taaaccatgg ccaagttgac cagtgccgtt ccggtgctca ccgcgcgcga cgtcgccgga |
| 4321 | gcggtcgagt tctggaccga ccggctcggg ttctcccggg acttcgtgga ggacgacttc |
| 4381 | gccggtgtgg tccgggacga cgtgaccctg ttcatcagcg cggtccagga ccaggtggtg |
| 4441 | ccggacaaca ccctggcctg ggtgtgggtg cgcggcctgg acgagctgta cgccgagtgg |
| 4501 | tcggaggtcg tgtccacgaa cttccgggac gcctccgggc cggccatgac cgagatcggc |
| 4561 | gagcagccgt gggggcggga gttcgccctg cgcgacccgg ccggcaactg cgtgcacttc |
| 4621 | gtggccgagg agcaggactg acacgtccga cggcggccca cgggtcccag gcctcggaga |
| 4681 | tccgtccccc ttttcctttg tcgatatcat gtaattagtt atgtcacgct tacattcacg |
| 4741 | ccctcccccc acatccgctc taaccgaaaa ggaaggagtt agacaacctg aagtctaggt |
| 4801 | ccctatttat ttttttatag ttatgttagt attaagaacg ttatttatat ttcaaatttt |
| 4861 | tctttttttt ctgtacagac gcgtgtacgc atgtaacatt atactgaaaa ccttgcttga |
| 4921 | gaaggttttg ggacgctcga aggctttaat ttgcaagctg gagaccaaca tgtgagcaaa |
| 4981 | aggccagcaa aaggccagga accgtaaaaa ggccgcgttg ctggcgtttt tccataggct |
| 5041 | ccgcccccct gacgagcatc acaaaaatcg acgctcaagt cagaggtggc gaaacccgac |
| 5101 | aggactataa agataccagg cgtttccccc tggaagctcc ctcgtgcgct ctcctgttcc |
| 5161 | gaccctgccg cttaccggat acctgtccgc ctttctccct tcgggaagcg tggcgctttc |
| 5221 | tcaatgctca cgctgtaggt atctcagttc ggtgtaggtc gttcgctcca agctgggctg |
| 5281 | tgtgcacgaa ccccccgttc agcccgaccg ctgcgcctta tccggtaact atcgtcttga |
| 5341 | gtccaacccg gtaagacacg acttatcgcc actggcagca gccactggta acaggattag |
| 5401 | cagagcgagg tatgtaggcg gtgctacaga gttcttgaag tggtggccta actacggcta |
| 5461 | cactagaagg acagtatttg gtatctgcgc tctgctgaag ccagttacct tcggaaaaag |
| 5521 | agttggtagc tcttgatccg gcaaacaaac caccgctggt agcggtggtt tttttgtttg |
| 5581 | caagcagcag attacgcgca gaaaaaaagg atctcaagaa gatcctttga tcttttctac |
| 5641 | ggggtctgac gctcagtgga acgaaaactc acgttaaggg attttggtca tgagatc |

The portion of the DNA sequence above that encodes the albumin binding peptide is shown in bold type. The underlined portion of the sequence above corresponds to NEP amino acids 47-749. The Kex2 protease cleaves between the alpha factor signal sequence and NEP (shown in bold), producing a mature form of the protease which is secreted into the media. Standard sequencing methods were used to verify that the desired construct, designated pPicZa-A-NEP, was obtained. The expressed protein is summarized as follows:

| | | |
|---|---|---|
| | DNA | |
| NAME | SEQUENCE | PROTEIN SEQUENCE |

| | | | |
|---|---|---|---|
| Alpha factor | 941-1195 | MRFPSIFTAVLFAASSALAAPVNTTTEDETAQIPAEAVIG | |
| signaling | | YSDLEGDFDVAVLPFSNSTNNGLLFINTTIASIAAKEEGV | |
| peptide | | SLEKR | |
| Albumin | 1196-1259 | DRLIEDICLPRWGCLWEDDGS | (SEQ ID NO: 6) |
| Binding | | | |
| Peptide | | | |
| Human NEP | 1260-3356 | Amino acids 47-749 of SWISSPROT ID: P08473 | |

The albumin binding peptide is also designed as a fusion protein to NEP^{(N144Q/N284Q/N310Q/N324Q/N334Q/N627Q)} and is shown in the construct below:

| (SEQ ID NO: 8) | |
|---|---|
| 1 | agatctaaca tccaaagacg aaaggttgaa tgaaaccttt ttgccatccg acatccacag |
| 61 | gtccattctc acacataagt gccaaacgca acaggagggg atacactagc agcagaccgt |
| 121 | tgcaaacgca ggacctccac tcctcttctc ctcaacaccc acttttgcca tcgaaaaacc |
| 181 | agcccagtta ttgggcttga ttggagctcg ctcattccaa ttccttctat taggctacta |
| 241 | acaccatgac tttattagcc tgtctatcct ggcccccctg gcgaggttca tgtttgttta |
| 301 | tttccgaatg caacaagctc cgcattacac ccgaacatca ctccagatga gggctttctg |
| 361 | agtgtggggt caaatagttt catgttcccc aaatggccca aaactgacag tttaaacgct |
| 421 | gtcttggaac ctaatatgac aaaagcgtga tctcatccaa gatgaactaa gtttggttcg |
| 481 | ttgaaatgct aacggccagt tggtcaaaaa gaaacttcca aaagtcggca taccgtttgt |
| 541 | cttgtttggt attgattgac gaatgctcaa aaataatctc attaatgctt agcgcagtct |
| 601 | ctctatcgct tctgaacccc ggtgcacctg tgccgaaacg caaatgggga aacacccgct |
| 661 | ttttggatga ttatgcattg tctccacatt gtatgcttcc aagattctgg tgggaatact |
| 721 | gctgatagcc taacgttcat gatcaaaatt taactgttct aacccctact tgacagcaat |
| 781 | atataaacag aaggaagctg ccctgtctta aacctttttt tttatcatca ttattagctt |
| 841 | actttcataa ttgcgactgg ttccaattga caagcttttg attttaacga cttttaacga |
| 901 | caacttgaga agatcaaaaa acaactaatt attcgaaacg atgagatttc cttcaatttt |
| 961 | tactgctgtt ttattcgcag catcctccgc attagctgct ccagtcaaca ctacaacaga |
| 1021 | agatgaaacg gcacaaattc cggctgaagc tgtcatcggt tactcagatt tagaagggga |
| 1081 | tttcgatgtt gctgttttgc cattttccaa cagcacaaat aacgggttat tgtttataaa |
| 1141 | tactactatt gccagcattg ctgctaaaga agaaggggta tctctcgaga aaagagaccg |
| 1201 | cctgatcgag gatatctgcc tgccccggtg gggctgcctg tgggaggatg atggtagtga |
| 1261 | tggtatttgc aagtcatcag actgcataaa atcagctgct cgactgatcc aaaacatgga |
| 1321 | tgccaccact gagccttgta cagacttttt caaatatgct tgcggaggct ggttgaaacg |
| 1381 | taatgtcatt cccgagacca gctcccgtta cggcaacttt gacattttaa gagatgaact |
| 1441 | agaagtcgtt ttgaaagatg tccttcaaga acccaaaact gaagatatag tagcagtgca |
| 1501 | gaaagcaaaa gcattgtaca ggtcttgtat aCAGgaatct gctattgata gcagaggtgg |
| 1561 | agaacctcta ctcaaactgt taccagacat atatgggtgg ccagtagcaa cagaaaactg |
| 1621 | ggagcaaaaa tatggtgctt cttggacagc tgaaaaagct attgcacaac tgaattctaa |
| 1681 | atatgggaaa aaagtcctta ttaatttgtt tgttggcact gatgataaga attctgtgaa |
| 1741 | tcatgtaatt catattgacc aacctcgact tggcctccct tctagagatt actatgaatg |
| 1801 | cactggaatc tataaagagg cttgtacagc atatgtggat tttatgattt ctgtggccag |
| 1861 | attgattcgt caggaagaaa gattgcccat cgatgaaaac cagcttgctt tggaaatgaa |
| 1921 | taaagttatg gaattggaaa aagaaattgc cCAGgctacg gctaaacctg aagatcgaaa |
| 1981 | tgatccaatg cttctgtata acaagatgac attggcccag atccaaaatC AGttttcact |
| 2041 | agagatcaat gggaagccat tcagctggtt gCAGttcaca aatgaaatca tgtcaactgt |
| 2101 | gCAGattagt attacaaatg aggaagatgt ggttgtttat gctccagaat atttaaccaa |
| 2161 | acttaagccc attcttacca aatattctgc cagagatctt caaaatttaa tgtcctggag |
| 2221 | attcataatg gatcttgtaa gcagcctcag ccgaacctac aaggagtcca gaaatgcttt |
| 2281 | ccgcaaggcc ctttatggta caacctcaga aacagcaact tggagacgtt gtgcaaacta |
| 2341 | tgtcaatggg aatatggaaa atgctgtggg gaggctttat gtggaagcag catttgctgg |
| 2401 | agagagtaaa catgtggtcg aggatttgat tgcacagatc cgagaagttt ttattcagac |
| 2461 | tttagatgac ctcacttgga tggatgccga gacaaaaaag agagctgaag aaaaggcctt |
| 2521 | agcaattaaa gaaaggatcg gctatcctga tgacattgtt tcaaatgata acaaactgaa |
| 2581 | taatgagtac ctcgagttga actacaaaga agatgaatac ttcgagaaca taattcaaaa |
| 2641 | tttgaaattc agccaaagta aacaactgaa gaagctccga gaaaaggtgg acaaagatga |
| 2701 | gtggataagt ggagcagctg tagtcaatgc attttactct tcaggaagaa atcagatagt |
| 2761 | cttcccagcc ggcattctgc agcccccctt ctttagtgcc cagcagtcca actcattgaa |
| 2821 | ctatgggggc atcggcatgg tcataggaca cgaaatcacc catggcttcg atgacaatgg |
| 2881 | cagaaacttt aacaaagatg gagacctcgt tgactggtgg actcaacagt ctgcaagtaa |
| 2941 | ctttaaggag caatcccagt gcatggtgta tcagtatgga CAGttttcct gggacctggc |
| 3001 | aggtggacag caccttaatg gaattaatac actgggagaa aacattgctg ataatggagg |
| 3061 | tcttggtcaa gcatacagag cctatcagaa ttatattaaa aagaatggcg aagaaaaatt |
| 3121 | acttcctgga cttgacctaa atcacaaaca actatttttc ttgaactttg cacaggtgtg |
| 3181 | gtgtggaacc tataggccag agtatgcggt taactccatt aaaacagatg tgcacagtcc |
| 3241 | aggcaatttc aggattattg ggactttgca gaactctgca gagttttcag aagcctttca |
| 3301 | ctgccgcaag aattcataca tgaatccaga aaagaagtgc cgggtttggt aataaccgcg |
| 3361 | gcggccgcca gctttctaga acaaaaactc atctcagaag aggatctgaa tagcgccgtc |
| 3421 | gaccatcatc atcatcatca ttgagtttgt agccttagac atgactgttc ctcagttcaa |
| 3481 | gttgggcact tacgagaaga ccggtcttgc tagattctaa tcaagaggat gtcagaatgc |
| 3541 | catttgcctg agagatgcag gcttcatttt tgatactttt ttatttgtaa cctatatagt |
| 3601 | ataggatttt ttttgtcatt ttgtttcttc tcgtacgagc ttgctcctga tcagcctatc |
| 3661 | tcgcagctga tgaatatctt gtggtagggg tttgggaaaa tcattcgagt ttgatgtttt |
| 3721 | tcttggtatt tcccactcct cttcagagta cagaagatta agtgagacct tcgtttgtgc |
| 3781 | ggatccccca cacaccatag cttcaaaatg tttctactcc ttttttactc ttccagattt |
| 3841 | tctcggactc cgcgcatcgc cgtaccactt caaaacaccc aagcacagca tactaaattt |
| 3901 | tccctctttc ttcctctagg gtgtcgttaa ttacccgtac taaaggtttg gaaaagaaaa |
| 3961 | aagagaccgc ctcgtttctt tttcttcgtc gaaaaaggca ataaaaattt ttatcacgtt |
| 4021 | tctttttctt gaaatttttt tttttagttt ttttctcttt cagtgacctc cattgatatt |
| 4081 | taagttaata aacggtcttc aatttctcaa gtttcagttt catttttctt gttctattac |
| 4141 | aacttttttt acttcttgtt cattagaaag aaagcatagc aatctaatct aaggggcggt |
| 4201 | gttgacaatt aatcatcggc atagtatatc ggcatagtat aatacgacaa ggtgaggaac |
| 4261 | taaaccatgg ccaagttgac cagtgccgtt ccggtgctca ccgcgcgcga cgtcgccgga |
| 4321 | gcggtcgagt tctggaccga ccggctcggg ttctcccggg acttcgtgga ggacgacttc |
| 4381 | gccggtgtgg tccgggacga cgtgaccctg ttcatcagcg cggtccagga ccaggtggtg |
| 4441 | ccggacaaca ccctggcctg ggtgtgggtg cgcggcctgg acgagctgta cgccgagtgg |
| 4501 | tcggaggtcg tgtccacgaa cttccgggac gcctccgggc cggccatgac cgagatcggc |
| 4561 | gagcagccgt gggggcggga gttcgccctg cgcgacccgg ccggcaactg cgtgcacttc |
| 4621 | gtggccgagg agcaggactg acacgtccga cggcggccca cgggtcccag gcctcggaga |
| 4681 | tccgtccccc ttttcctttg tcgatatcat gtaattagtt atgtcacgct tacattcacg |
| 4741 | ccctcccccc acatccgctc taaccgaaaa ggaaggagtt agacaacctg aagtctaggt |
| 4801 | ccctatttat ttttttatag ttatgttagt attaagaacg ttatttatat ttcaaatttt |
| 4861 | tctttttttt ctgtacagac gcgtgtacgc atgtaacatt atactgaaaa ccttgcttga |
| 4921 | gaaggttttg ggacgctcga aggctttaat ttgcaagctg gagaccaaca tgtgagcaaa |
| 4981 | aggccagcaa aaggccagga accgtaaaaa ggccgcgttg ctggcgtttt tccataggct |
| 5041 | ccgcccccct gacgagcatc acaaaaatcg acgctcaagt cagaggtggc gaaacccgac |
| 5101 | aggactataa agataccagg cgtttccccc tggaagctcc ctcgtgcgct ctcctgttcc |
| 5161 | gaccctgccg cttaccggat acctgtccgc ctttctccct tcgggaagcg tggcgctttc |
| 5221 | tcaatgctca cgctgtaggt atctcagttc ggtgtaggtc gttcgctcca agctgggctg |
| 5281 | tgtgcacgaa ccccccgttc agcccgaccg ctgcgcctta tccggtaact atcgtcttga |
| 5341 | gtccaacccg gtaagacacg acttatcgcc actggcagca gccactggta acaggattag |
| 5401 | cagagcgagg tatgtaggcg gtgctacaga gttcttgaag tggtggccta actacggcta |
| 5461 | cactagaagg acagtatttg gtatctgcgc tctgctgaag ccagttacct tcggaaaaag |
| 5521 | agttggtagc tcttgatccg gcaaacaaac caccgctggt agcggtggtt tttttgtttg |
| 5581 | caagcagcag attacgcgca gaaaaaaagg atctcaagaa gatcctttga tcttttctac |
| 5641 | ggggtctgac gctcagtgga acgaaaactc acgttaaggg attttggtca tgagatc |

The portion of the DNA sequence above that encodes the albumin binding peptide is shown in bold type. The underlined portion of the sequence above corresponds to NEP amino acids 47-749. The Kex2 protease cleaves between the alpha factor signal sequence and NEP (shown in bold), producing a mature form of the protease which is secreted into the media. Standard sequencing methods were used to verify that the desired construct, designated pPicZa-A-NEP, was obtained. The expressed protein is summarized as follows:

| | DNA | |
|---|---|---|
| NAME | SEQUENCE | PROTEIN SEQUENCE |
| Alpha factor | 941-1195 | MRFPSIFTAVLFAASSALAAPVNTTTEDETAQIPAEAVIG |
| signaling | | YSDLEGDFDVAVLPFSNSTNNGLLFINTTIASIAAKEEGV |
| peptide | | SLEKR |
| Albumin | 1196-1259 | DRLIEDICLPRWGCLWEDDGS |
| Binding | | |
| Peptide | | |
| Human NEP | 1260-3356 | Amino acids 47-749 of SWISSPROT ID: P08473 |
| | | N144Q, N284Q, N310Q, N324Q, N334Q, |
| | | and N627Q |

### Example 6

### Demonstration of Efficacy in Animal Models of Inflammatory Bowel Disease

A. TNBS Induced Colitis Model

In this validated experimental model of colitis, TNBS (2,4,6 trinitrobenzene-sulfonic acid, Sigma) is added to mice at a dose of approximately 2-6 mg per mouse, via rectal injection anesthetized with Enflurane, to induce severe, transmural Th1 mediated colitis. Recombinant NEP, for example, as produced in Example A, can be administered by a variety of routes at various doses to treat this induced colitis in accordance with the methods of the invention. The effect of administrating recombinant NEP on TNBS induced colitis can be measured by the following scores: macroscopic, histologic, and myeloperoxidase activity. For macroscopic damage, tissue from the proximal colon is removed at various time points and immediately scored. For histological examination, the tissue is fixed in 10% formalin, then stained with either hematoxylin or eosin, and then scored for inflammation. To test for granulocyte infiltration myeloperoxidase activity, a commercially available kit is employed, and the readout is in units/mg tissue. A positive result is described as follows. After onset of TNBS induced colitis in this model, administration of recombinant NEP results in a decrease in macroscopic score, histologic score, and myeloperoxidase activity when compared to parallel administration of a control protein such as serum albumin in a seperate mouse.

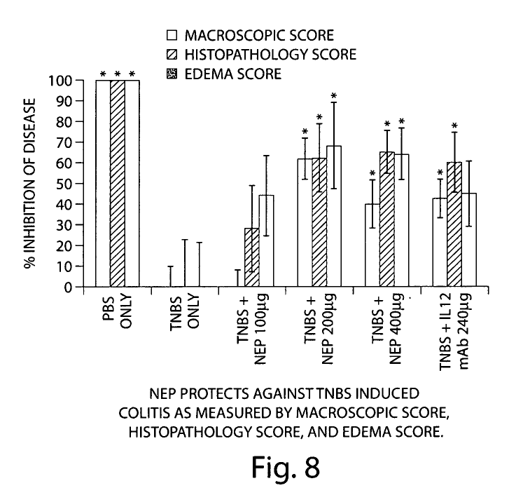

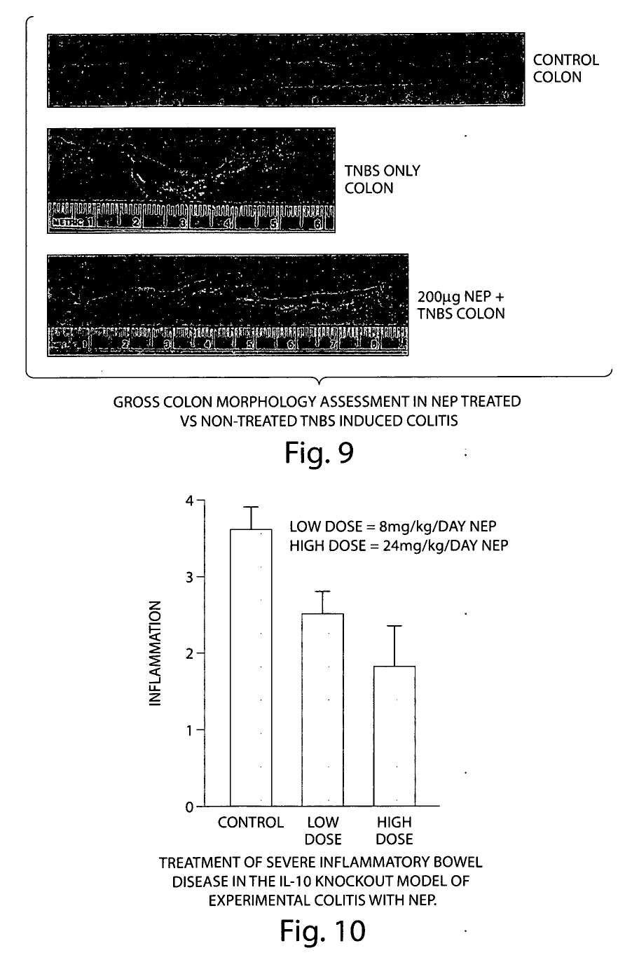

The recombinant, truncated human NEP of the present invention is administered to mice having TNBS induced colitis and its therapeutic effect demonstrated by a decrease in the macroscopic score, histologic score, and observed myeloperoxidase activity. Therapeutically effective administration of 0.3, 1.0, 3, 10, and 20 mg/kg of active, recombinant, truncated human NEP is employed to find the optimal dose that maximizes the desired therapeutic effect with minimal toxic side effects in this model (for a mouse of average weight, ˜25 grams, the dose is ˜7.5, 25, 75, 250, and 500 μg, per mouse, respectively, at these doses). Recombinant human truncated NEP shows potent anti-inflammatory activity in the TNBS model of acute colitis as show in FIG. 7-9 when tested at a single topically administered dose of 200 or 400 μg/mouse. FIG. 7 shows that TNBS induced colitis causes a dramatic weight loss (approximately 10% body weight loss in 3 days) which can be protected by the administration of 200 or 400 μg of NEP per mouse. The percent inhibition of disease in TNBS colitis was judged by histopathological, macroscopic, and edema score. At doses of 200 μg/mouse, NEP inhibited greater than 60% of the disease as judged by three scoring criteria. The gross colon morphology assement shows that TNBS treated colons are significantly shrunken and thicker (FIG. 9). When treated with 200 μg of NEP, thse colons are protected against the TNBS induced colon damage (FIG. 9). Buffer-only controls and are included to demonstrate that elimination of colitis is dependent on the administration of NEP.

B. IL-10 Knockout Model

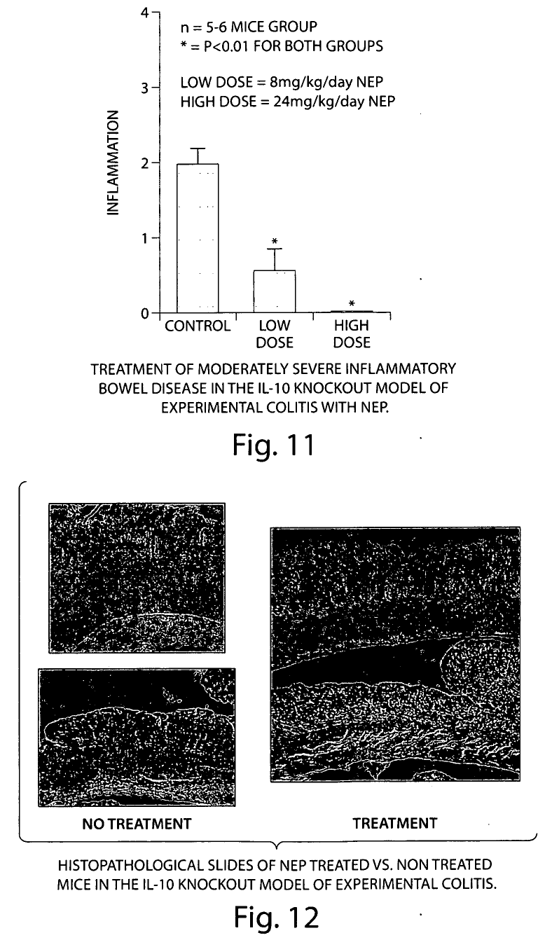

Human patients with IBD tend to have a low Interleukin-10 producer genotype more often than normal controls. Mice lacking specific components of the immune response, such as IL-10, IL-2, or the receptor chains of T-cells, spontaneously develop bowel inflammation. Mice with allele specific knockouts of IL-10 develop a spontaneous inflammation that resembles Crohn's Disease. IL-10 knockout mice are commercially available from Harlan UK. The effect of administration of recombinant, truncated human NEP, and other NEPs of the invention, on inflammation can be demonstrated by histological score and level of cytokines in stool, which correlate with activity of bowel inflammation. To induce colitis, mice 4-5 wk of age were given piroxicam (Sigma-Aldrich, St. Louis, Mo.) mixed into their feed (National Institutes of Health-31M) for 2 wk. They received 60 mg of piroxicam/250 g of food wk1 and 80 mg piroxicam/250 g of food wk 2. Mice subsequently were placed on the normal rodent chow without piroxicam. The colitis was evaluated from 2-16 days after colitis induction. Mice were given NEP at 8 and 24 mg/kg/day for 2 wk tatting 2 days after discontinuation of the piroxicam. NEP was given by continuous SQ infusion using osmotic pumps (Alzet, Cupertino, Calif.). Control mice also had implantation of osmotic pumps releasing just control buffer. For the histological score, samples from the colon are graded on the number of observed lesions, which is a measure of the degree of inflammation caused by the IL-10 knockout phenotype. A high degree of intestinal inflammation produces a large number of lesions and increased cytokine level in stool samples. Therapeutically effective administration of 0.3, 1.0, 3, 10 or 25 mg/kg of active, recombinant, truncated human NEP is employed to find the optimal dose that maximizes the desired therapeutic effect, ie attenuated inflammation, with minimal toxic side effects in this model (for a mouse of average weight, ˜25 grams, the dose is ˜7.5, 25, 75, and 250 μg, per mouse, respectively, at these doses). The dosing can also occur over the course of two weeks via osmotic pump delivery. In this example, NEP was dosed at 8 and 24 mg/kg/day for 2 weeks via pump delivery. A positive therapeutic effect would result in a decreased histological score and upon addition of recombinant NEP. This is shown in FIGS. 10, 11, and 12 show that NEP has a inhibits inflammation in this model of colitis. To assess Colons (from the ileocecal value to the mid descending colon) were opened longitudinally and rolled up onto a glass rod. The tissue was fixed in 10% neutral buffered formalin, removed from the glass rods without unrolling the tissue, and processed for sectioning. Tissue was sliced to obtain longitudinal sections of colon that were 6 μm thick and then stained with H&E for light microscopic examination. The inflammation was scored from 0-4 using the following criteria: grade 0, no change from normal tissue; grade 1, patchy mononuclear cell infiltrates in the LP; grade 2, more uniform mononuclear cell inflammation involving both the epithelium and LP; this was accompanied by minimal epithelial hyperplasia and slight to no depletion of mucous from goblet cells; grade 3, some epithelial and muscle hypertrophy with patchy lymphocytic infiltrates extending into the muscle layers; there were mucus depletion and occasional crypt abscesses and epithelial erosions; and grade 4, lesions involved most of the intestinal section. The inflammation, which was comprised mostly of lymphocytes and some neutrophils, was transmural and severe. There was prominent thickening of both the epithelial and muscle layers. There was mucus depletion and more frequent crypt abscesses. Ulcerations were frequent.

### Example 7

### The SAMP/YIT Model of Spontaneous Colitis

Description of ileitis in SAMP1/Yit mice. Mild to moderate ileitis was first found in SAMP1/Yit mice by 20 weeks of age, and reached 100% penetrance by 30 weeks. Lesion severity and incidence increased with age. Histologicalanalysis of stomach, liver, kidney, spleen, mesenteric and peripheral lymph nodes, and thymus revealed no significant extraintestinal inflammation. SAMP1/Yit mice exhibited discontinuous areas of transmural intestinal inflammation, most severe in the terminal ileum Severe inflammatory lesions could be identified by visual inspection as discrete areas of bowel wall thickening and relative stenosis of the lumen. Histological examination revealed mononuclear and PMN cell infiltrates in the lamina propria, submucosa, and muscle layers. PMNs were found most abundantly in the lamina propria and submucosa. There was focal infiltration of the epithelium by PMNs to form lesions of active cryptitis and crypt microabscesses, identical to the lesions found in human CD. Many but not all of the inflammatory lesions were associated with Peyer's patches. Early inflammatory lesions consisting of neutrophils causing epithelial damage overlying these preexisting lymphoid aggregates were commonly identified, resembling the aphthoid lesions found in human CD. Mucosal ulceration and intestinal fistulae were uncommon. The mononuclear cell population consisted of cells morphologically compatible with histiocytes (tissue macrophages), lymphocytes, and plasma cells. Abnormal accumulations of plasma cells could be seen at the base of the mucosa in chronically inflamed areas, compatible with the basal plasmacytosis seen in human chronic inflammatory bowel disease. In some animals, the tissue macrophages focally coalesced into loose aggregates compatible with granuloma formation. The normal delicate villous architecture, with a villus/crypt ratio of 4:1 to 5:1, was lost in inflammatory lesions to various degrees by a combination of elongation of the crypts and expansion of the lamina propria. This combination of changes lead to areas in the majority of animals that, although not reduced in overall mucosal height, nevertheless had complete loss of villous architecture. In older mice, the severely inflamed areas also showed prominent mucosal fibrosis and distortion of the normally straight crypt architecture in the form of budded and branched glands. In chronically inflamed areas, changes in epithelial phenotype were typically observed in the form of Paneth cell and goblet cell hyperplasia. The “pyloric metaplasia” commonly seen in human CD was not observed in these animals. Other histological features observed in these mice in common with human CD included muscular and neural hyperplasia of the bowel wall and mucosal lymphangiectasia. Although the colons of these animals never developed grossly identifiable inflammatory lesions, histological examination showed focal areas of mucosal and transmural inflammation in some animals, always of lesser severity than the inflammation found in the terminal ileum. The overall pathological assessment of these animals was a disease process remarkably similar to that seen in human CD

NEP is tested in this model as follows: NEP is administered in a 7 day or 2 week subcutaneous osmotic pump to 40-week-old SAMP1/YitFc mice. Control animals are age-matched SAMP1/YitFc mice treated with saline only pumps. All animals are sacrificed at 7 or 14 days after treatment. The histological assessment of the colons is described as above for the IL-10 knockout model.

### LIST OF REFERENCES CITED

1. Otsuka M and Yoshioka K. Neurotransmitter functions of mammalian tachykinins. Physiol Rev 73: 229-308, 1993.
2. McDonald D M, Bowden J J, Baluk P, and Bunnett N W. Neurogenic inflammation. A model for studying efferent actions of sensory nerves. Adv Exp Med Biol 410: 453-462, 1996.
3. McDonald D M, Bowden J J, Baluk P, and Bunnett N W. Neurogenic inflammation. A model for studying efferent actions of sensory nerves. Adv Exp Med Biol 410: 453-462, 1996.
4. Kirkwood K S, Bunnett N W, Maa J, Castagliolo I, Liu B, Gerard N, Zacks J, Pothoulakis C, and Grady E F. Deletion of neutral endopeptidase exacerbates intestinal inflammation induced by Clostridium difficile toxin A. Am J Physiol Gastrointest Liver Physiol 281: G544-551., 2001.
5. Sturiale S, Barbara G, Qiu B, Figini M, Geppetti P, Gerard N, Gerard C, Grady E F, Bunnett N W, and Collins S M. Neutral endopeptidase (EC 3.4.24.11) terminates colitis by degrading substance P. Proc Natl Acad Sci USA 96: 11653-11658., 1999.
6. Scholzen T E, Steinhoff M, Bonaccorsi P, Klein R, Amadesi S, Geppetti P, Lu B, Gerard N P, Olerud J E, Luger T A, Bunnett N W, Grady E F, Armstrong C A, and Ansel J C. Neutral endopeptidase terminates substance P-induced inflammation in allergic contact dermatitis. J Immunol 166: 1285-1291, 2001
7. Mantyh C R, Maggio J E, Mantyh P W, Vigna S R, and Pappas T N. Increased substance P receptor expression by blood vessels and lymphoid aggregates in Clostridium difficile-induced pseudomembranous colitis. Dig Dis Sci 41: 614-620, 1996.
8. Mantyh C R, Gates T S, Zimmerman R P, Welton W L, Passaro E P, Vigna S R, Maggio J E, Kruger L, and Mantyh P W. Receptor binding sites for substance P, but not substance K or neuromedin are expressed in high concentrations by arterioles, venules, and lymph nodules in surgical specimens obtained from patients with ulcerative colitis and Crohn's disease. Proc Natl Acad Sci USA 85: 3235-3239, 1988.
9. Mantyh C R, Vigna S R, Bollinger R R, Mantyh P W, Maggio J E, and Pappas T N. Differential expression of substance P receptors in patients with Crohn's disease and ulcerative colitis. Gastroenterology 109: 850-860, 1995.
10. Pothoulakis C, Castagliuolo I, LaMont J T, Jaffer A, O'Keane J C, Snider R M, and Leeman S E. CP-96,345, a substance P antagonist, inhibits rat intestinal responses to Clostridium difficile toxin A but not cholera toxin. Proc Natl Acad Sci USA 91: 947-951, 1994.
11. Sturiale S, Barbara G, Qiu B, Figini M, Geppetti P, Gerard N, Gerard C, Grady E F, Bunnett N W, and Collins S M. Neutral endopeptidase (EC 3.4.24.11) terminates colitis by degrading substance P. Proc Natl Acad Sci USA 96: 11653-11658, 1999
12. Roques B P, Noble F, Dauge V, Fournie-Zaluski M C, and Beaumont A. Neutral endopeptidase 24.11: structure, inhibition, and experimental and clinical pharmacology. Pharmacol Rev 45: 87-146, 1993.
13. Dennis M S, Zhang M, Meng Y G, Kadkhodayan M, Kirchhofer D, Combs D, Damico L A. Albumin binding as a general strategy for improving the pharmacokinetics of proteins. J Biol. Chem. 2002 Sep. 20;277(38):35035-43.

### Equivalents

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications considered to be within the scope of the following claims.

1. A method for making a recombinant, truncated mammalian neutral endopeptidase (NEP), said method comprising culturing a host cell that comprises a nucleic acid vector encoding a truncated mammalian NEP.

2. The method of claim 1, wherein said truncated mammalian NEP comprises amino acids 47-749 of SEQ ID NO: 2.

3. The method of claim 1, wherein said truncated mammalian NEP consists of amino acids 47-749 of SEQ ID NO: 2.

4. The method of claim 1, wherein said vector is pPicZα-A-NEP.

5. A method for purifying a recombinant, truncated mammalian neutral endopeptidase (NEP), said method comprising adding about 60% ammonium sulfate to a solution comprising said NEP, removing any precipitate, if present, from said solution, and subjecting said solution to chromatography comprising hydrophobic interaction chromatography and anion exchange chromatography.

6. The purified recombinant, truncated mammalian NEP obtained by the method of claim 5, wherein said NEP is more than 95% pure.

7. A pharmaceutical formulation comprising recombinant, truncated mammalian neutral endopeptidase (NEP) comprising amino acids 47-749 of SEQ ID NO: 2.

8. The pharmaceutical formulation of claim 7, wherein said NEP is encapsulated in an enteric coating.

9. A method for treating inflammatory bowel disease in a mammalian subject in need thereof, said method comprising administering to said subject a therapeutically effective dose of a recombinant, truncated mammalian neutral endopeptidase (NEP) or a bacterial homolog of said NEP.

10. The method of claim 9, wherein said NEP comprises amino acids 47-749 of SEQ ID NO: 2.

11. The method of claim 9, wherein said NEP is administered at a dose ranging from 0.1 mg of NEP per kg of said subject's body weight to 10 mg/kg of NEP per kg of said subject's body weight.

12. A method for preventing or reducing a symptom of inflammatory bowel disease in a mammalian subject, said method comprising the steps of:
a) identifying a mammalian subject at risk of inflammatory bowel disease; and
b) administering to said subject a therapeutically effective dose of a recombinant, truncated mammalian neutral endopeptidase (NEP) or a bacterial homolog thereof.

13. The method of claim 12, wherein said subject is human and is identified on the basis of family history or prior history of inflammatory bowel disease.

14. The method of claim 12, wherein said NEP is administered to said subject prior to the onset of one or more symptoms of inflammatory bowel disease.

15. A pharmaceutical composition comprising in a unit dose, from about 1 to about 200 mg of a truncated NEP or an NEP homolog and a pharmaceutically acceptable excipient or carrier.

16. The pharmaceutical composition of claim 16, wherein said unit dose consists of between 20 and 100 mg of said truncated NEP or NEP homolog.

17. A method of treatment of inflammatory bowel disease in a human patient suffering therefrom, said method comprising administering to said human a unit dose of truncated NEP, wherein said unit dose consists of between 20 and 100 mg of said truncated NEP or NEP homolog.

18. The method of claim 17, wherein said NEP comprises amino acids 47-749 of SEQ ID NO: 2.

19. The method of claim 17, wherein said NEP comprises amino acids 47-749 of SEQ ID NO: 2, wherein one or more asparagine residues in SEQ ID NO: 2 are replaced by one or more amino acids other than asparagine.

20. The method of claim 17, wherein said NEP comprises amino acids 47-749 of SEQ ID NO: 2, wherein N144, N284, N310, N324, N334, and N627 are replaced by one or more amino acids other than asparagine.

21. The method of claim 18, wherein said dose is administered by infusion.
